(19) United States
(12) Patent Application Publication
(10) Pub. No.: US 2009/0285774 A1
(43) Pub. Date: Nov. 19, 2009

(54) Hepatitis C Virus Inhibitors

(75) Inventors: Ny Sin, East Hampton, CT (US); Brian Lee Venables, Durham, CT (US); Paul Michael Scola, Glastonbury, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/465,142

(22) Filed: May 13, 2009

Related U.S. Application Data
(60) Provisional application No. 61/053,477, filed on May 15, 2008.

(51) Int. Cl. A61K 38/20; C07K 5/08; A61K 38/06; A61K 38/21; A61P 31/12

(52) U.S. Cl. 424/85.2; 530/331; 514/18; 424/85.4; 424/85.7

(57) ABSTRACT

Hepatitis C virus inhibitors having the general formula

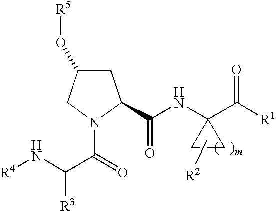

are disclosed. Compositions comprising the compounds and methods for using the compounds to inhibit HCV are also disclosed.

### CROSS-REFERENCE TO RELATED APPLICATIONS

This application claims the benefit of U.S. Provisional Application Ser. No. 61/053,477 filed May 15, 2008.

The present disclosure is generally directed to antiviral compounds, and more specifically directed to compounds which inhibit the function of the NS3 protease (also referred to herein as “serine protease”) encoded by Hepatitis C virus (HCV), compositions comprising such compounds, and methods for inhibiting the function of the NS3 protease.

HCV is a major human pathogen, infecting an estimated 170 million persons worldwide—roughly five times the number infected by human immunodeficiency virus type 1. A substantial fraction of these HCV infected individuals develop serious progressive liver disease, including cirrhosis and hepatocellular carcinoma.

Presently, the most effective HCV therapy employs a combination of alpha-interferon and ribavirin, leading to sustained efficacy in 40% of patients. Recent clinical results demonstrate that pegylated alpha-interferon is superior to unmodified alpha-interferon as monotherapy. However, even with experimental therapeutic regimens involving combinations of pegylated alpha-interferon and ribavirin, a substantial fraction of patients do not have a sustained reduction in viral load. Thus, there is a clear and unmet need to develop effective therapeutics for treatment of HCV infection.

HCV is a positive-stranded RNA virus. Based on a comparison of the deduced amino acid sequence and the extensive similarity in the 5′ untranslated region, HCV has been classified as a separate genus in the Flaviviridae family. All members of the Flaviviridae family have enveloped virions that contain a positive stranded RNA genome encoding all known virus-specific proteins via translation of a single, uninterrupted, open reading frame.

Considerable heterogeneity is found within the nucleotide and encoded amino acid sequence throughout the HCV genome. Six major genotypes have been characterized, and more than 50 subtypes have been described. The major genotypes of HCV differ in their distribution worldwide, and the clinical significance of the genetic heterogeneity of HCV remains elusive despite numerous studies of the possible effect of genotypes on pathogenesis and therapy.

The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins. In the case of HCV, the generation of mature non-structural proteins (NS2, NS3, NS4A, NS4B, NS5A, and NS5B) is effected by two viral proteases. The first one cleaves at the NS2-NS3 junction; the second one is a serine protease contained within the N-terminal region of NS3 and mediates all the subsequent cleavages downstream of NS3, both in cis, at the NS3-NS4A cleavage site, and in trans, for the remaining NS4A-NS4B, NS4B-NS5A, NS5A-NS5B sites. The NS4A protein appears to serve multiple functions, acting as a co-factor for the NS3 protease and possibly assisting in the membrane localization of NS3 and other viral replicase components. The complex formation of the NS3 protein with NS4A is essential for efficient polyprotein processing, enhancing the proteolytic cleavage at all of the sites. The NS3 protein also exhibits nucleoside triphosphatase and RNA helicase activities. NS5B is a RNA-dependent RNA polymerase that is involved in the replication of HCV.

The present disclosure provides peptide compounds that can inhibit the functioning of the NS3 protease, e.g., in combination with the NS4A protease. Further, the present disclosure describes the administration of combination therapy to a patient whereby a compound in accordance with the present disclosure, which is effective to inhibit the HCV NS3 protease, can be administered with one or two additional compounds having anti-HCV activity.

In a first aspect the present disclosure provides a compound of Formula (I)

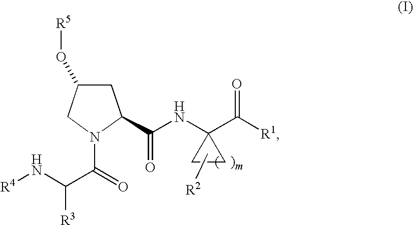

or a pharmaceutically acceptable salt thereof, wherein

m is 1, 2, or 3;

R¹ is selected from hydroxy and —NHSO₂R⁶; wherein R⁶ is selected from alkyl, aryl, cycloalkyl, (cycloalkyl)alkyl, heterocyclyl, and —NR^{a}R^{b}, wherein the alkyl, the cycloalkyl and the cycloalkyl part of the (cycloalkyl)alkyl are optionally substituted with one, two, or three substituents selected from alkenyl, alkoxy, alkoxyalkyl, alkyl, arylalkyl, arylcarbonyl, cyano, cycloalkenyl, (cycloalkyl)alkyl, halo, haloalkoxy, haloalkyl, and (NR^{e}R^{f})carbonyl;

R² is selected from hydrogen, alkenyl, alkyl, and cycloalkyl, wherein the alkenyl, alkyl, and cycloalkyl are optionally substituted with halo;

R³ is selected from alkenyl, alkoxyalkyl, alkoxycarbonylalkyl, alkyl, arylalkyl, carboxyalkyl, cyanoalkyl, cycloalkyl, (cycloalkyl)alkyl, haloalkoxy, haloalkyl, (heterocyclyl)alkyl, hydroxyalkyl, (NR^{c}R^{d})alkyl, and (NR^{e}R^{f})carbonylalkyl;

R⁴ is selected from phenyl and a five- or six-membered partially or fully unsaturated ring optionally containing one, two, three, or four heteroatoms selected from nitrogen, oxygen, and sulfur; wherein each of the rings is optionally substituted with one, two, three, or four substitutents independently selected from alkoxy, alkoxycarbonyl, alkyl, alkylcarbonyl, alkylsulfanyl, carboxy, cyano, cycloalkyl, cycloalkyloxy, halo, haloalkyl, haloalkoxy, —NR^{c}R^{d}, (NR^{e}R^{f})carbonyl, (NR^{e}R^{f})sulfonyl, and oxo; provided that when R⁴ is a six-membered substituted ring all substituents on the ring other than fluoro must be in the meta and/or para positions relative to the ring's point of attachment to the parent molecular moiety;

R⁵ is selected from alkylcarbonyl, aryl, arylalkyl, arylalkylcarbonyl, arylcarbonyl, heterocyclyl, heterocyclylalkyl, heterocyclylalkylcarbonyl, heterocyclylcarbonyl, and (NR^{g}R^{h})carbonyl, wherein the aryl; the aryl part of the arylalkyl, the arylalkylcarbonyl, and the arylcarbonyl; the heterocycyl; and the heterocyclyl part of the heterocyclylalkyl and the heterocyclylalkylcarbonyl are each optionally substituted with from one to six R⁷ groups; provided that when R⁵ is heterocyclyl the heterocyclyl is other than

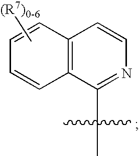

each R⁷ is independently selected from alkoxy, alkoxycarbonyl, alkyl, alkylcarbonyl, aryl, carboxy, cyano, cyanoalkyl, cycloalkyl, halo, haloalkyl, haloalkoxy, heterocyclyl, hydroxy, hydroxyalkyl, nitro, —NR^{c}R^{d}, (NR^{c}R^{d})alkyl, (NR^{c}R^{d})alkoxy, (NR^{e}R^{f})carbonyl, and (NR^{e}R^{f})sulfonyl; or

two adjacent R⁷ groups, together with the carbon atoms to which they are attached, form a four- to seven-membered partially- or fully-unsaturated ring optionally containing one or two heteroatoms independently selected from nitrogen, oxygen, and sulfur, wherein the ring is optionally substituted with one, two, or three groups independently selected from alkoxy, alkyl, cyano, halo, haloalkoxy, and haloalkyl;

R^{a} and R^{b} are independently selected from hydrogen, alkoxy, alkyl, aryl, arylalkyl, cycloalkyl, (cycloalkyl)alkyl, heterocyclyl, and heterocyclylalkyl; or R^{a} and R^{b} together with the nitrogen atom to which they are attached form a four to seven-membered monocyclic heterocyclic ring;

R^{c} and R^{d} are independently selected from hydrogen, alkoxyalkyl, alkoxycarbonyl, alkyl, alkylcarbonyl, arylalkyl, and haloalkyl;

R^{e} and R^{f} are independently selected from hydrogen, alkyl, aryl, arylalkyl, and heterocyclyl; wherein the aryl, the aryl part of the arylalkyl, and the heterocyclyl are optionally substituted with one or two substituents independently selected from alkoxy, alkyl, and halo; and

R^{g} and R^{h} are independently selected from hydrogen, alkyl, aryl, arylalkyl, cycloalkyl, (cycloalkyl)alkyl, heterocyclyl, and heterocyclyl; or R^{g} and R^{h} together with the nitrogen atom to which they are attached form a monocyclic heterocyclic ring wherein the monocyclic heterocyclic ring is optionally fused to a phenyl ring to form a bicyclic system; wherein the monocyclic heterocyclic ring and the bicyclic system are optionally substituted with one, two, or three substituents independently selected from alkoxy, alkyl, halo, haloalkoxy, and haloalkyl.

In a first embodiment of the first aspect, the present disclosure provides a compound of Formula (I), or a pharmaceutically acceptable salt thereof, wherein R¹ is —NHSO₂R⁶.

In a second embodiment of the first aspect, the present disclosure provides a compound of Formula (I), or a pharmaceutically acceptable salt thereof, wherein

m is 1 or 2;

R¹ is —NHSO₂R⁶; wherein R⁶ is selected from alkyl, aryl, cycloalkyl, (cycloalkyl)alkyl, heterocyclyl, and —NR^{a}R^{b}, wherein the alkyl, the cycloalkyl and the cycloalkyl part of the (cycloalkyl)alkyl are optionally substituted with one, two, or three substituents selected from alkenyl, alkoxy, alkoxyalkyl, alkyl, arylalkyl, arylcarbonyl, cyano, cycloalkenyl, (cycloalkyl)alkyl, halo, haloalkoxy, haloalkyl, and NR^{e}R^{f})carbonyl;

R² is selected from alkenyl, alkyl, and cycloalkyl, wherein the alkenyl, alkyl, and cycloalkyl are optionally substituted with halo;

R³ is selected from alkenyl and alkyl;

R⁴ is selected from phenyl and a five- or six-membered partially or fully unsaturated ring optionally containing one, two, three, or four heteroatoms selected from nitrogen, oxygen, and sulfur; wherein each of the rings is optionally substituted with one, two, three, or four substitutents independently selected from alkoxy, alkoxycarbonyl, alkyl, alkylcarbonyl, alkylsulfanyl, carboxy, cyano, cycloalkyl, cycloalkyloxy, halo, haloalkyl, haloalkoxy, —NR^{c}R^{d}, (NR^{e}R^{f})carbonyl, (NR^{e}R^{f})sulfonyl, and oxo; provided that when R⁴ is a six-membered substituted ring all substituents on the ring other than fluoro must be in the meta and/or para positions relative to the ring's point of attachment to the parent molecular moiety;

R⁵ is selected from alkylcarbonyl, aryl, arylalkyl, arylalkylcarbonyl, arylcarbonyl, heterocyclyl, heterocyclylalkyl, heterocyclylalkylcarbonlyl, heterocyclylcarbonyl, and (NR^{g}R^{h})carbonyl, wherein the aryl; the aryl part of the arylalkyl, the arylalkylcarbonyl, and the arylcarbonyl; the heterocycyl; and the heterocyclyl part of the heterocyclylalkyl and the heterocyclylalkylcarbonyl are each optionally substituted with from one to six R⁷ groups; provided that when R⁵ is heterocyclyl the heterocyclyl is other than

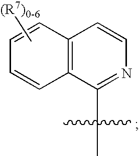

each R⁷ is independently selected from alkoxy, alkoxycarbonyl, alkyl, alkylcarbonyl, aryl, carboxy, cyano, cyanoalkyl, cycloalkyl, halo, haloalkyl, haloalkoxy, heterocyclyl, hydroxy, hydroxyalkyl, nitro, —NR^{c}R^{d}, (NR^{c}R^{d})alkyl, (NR^{c}R^{d})alkoxy, (NR^{e}R^{f})carbonyl, and (NR^{e}R^{f})sulfonyl; or

two adjacent R⁷ groups, together with the carbon atoms to which they are attached, form a four- to seven-membered partially- or fully-unsaturated ring optionally containing one or two heteroatoms independently selected from nitrogen, oxygen, and sulfur, wherein the ring is optionally substituted with one, two, or three groups independently selected from alkoxy, alkyl, cyano, halo, haloalkoxy, and haloalkyl;

R^{a} and R^{b} are independently selected from hydrogen, alkoxy, alkyl, aryl, arylalkyl, cycloalkyl, (cycloalkyl)alkyl, heterocyclyl, and heterocyclylalkyl; or R^{a} and R^{b} together with the nitrogen atom to which they are attached form a four- to seven-membered monocyclic heterocyclic ring;

R^{c} and R^{d} are independently selected from hydrogen, alkoxyalkyl, alkoxycarbonyl, alkyl, alkylcarbonyl, arylalkyl, and haloalkyl;

R^{e} and R^{f} are independently selected from hydrogen, alkyl, aryl, arylalkyl, and heterocyclyl; wherein the aryl, the aryl part of the arylalkyl, and the heterocyclyl are optionally substituted with one or two substituents independently selected from alkoxy, alkyl, and halo; and

R^{g} and R^{h} are independently selected from hydrogen, alkyl, aryl, arylalkyl, cycloalkyl, (cycloalkyl)alkyl, heterocyclyl, and heterocyclyl; or R^{g} and R^{h} together with the nitrogen atom to which they are attached form a monocyclic heterocyclic ring wherein the monocyclic heterocyclic ring is optionally fused to a phenyl ring to form a bicyclic system; wherein the monocyclic heterocyclic ring and the bicyclic system are optionally substituted with one, two, or three substituents independently selected from alkoxy, alkyl, halo, haloalkoxy, and haloalkyl.

In a third embodiment of the first aspect the present disclosure provides a compound of Formula (I), or a pharmaceutically acceptable salt thereof, wherein

m is 1 or 2;

R¹ is —NHSO₂R⁶; wherein R⁶ is unsubstituted cycloalkyl;

R² is selected from alkenyl, alkyl and cycloalkyl, wherein the alkenyl, alkyl, and cycloalkyl are optionally substituted with halo;

R³ is selected from alkenyl and alkyl;

R⁴ is selected from phenyl and a five- or six-membered partially or fully unsaturated ring optionally containing one, two, three, or four heteroatoms selected from nitrogen, oxygen, and sulfur; wherein each of the rings is optionally substituted with one, two, three, or four substitutents independently selected from alkoxy, alkoxycarbonyl, alkyl, alkylcarbonyl, alkylsulfanyl, carboxy, cyano, cycloalkyl, cycloalkyloxy, halo, haloalkyl, haloalkoxy, —NR^{c}R^{d}, (NR^{e}R^{f})carbonyl, NR^{e}R^{f})sulfonyl, and oxo; provided that when R⁴ is a six-membered substituted ring all substituents on the ring other than fluoro must be in the meta and/or para positions relative to the ring's point of attachment to the parent molecular moiety;

R⁵ is selected from alkylcarbonyl, aryl, arylalkyl, arylalkylcarbonyl, arylcarbonyl, heterocyclyl, heterocyclylalkyl, heterocyclylalkylcarbonyl, heterocyclylcarbonyl, and (R^{g}R^{h})carbonyl, wherein the aryl; the aryl part of the arylalkyl, the arylalkylcarbonyl, and the arylcarbonyl; the heterocycyl; and the heterocyclyl part of the heterocyclylalkyl and the heterocyclylalkylcarbonyl are each optionally substituted with from one to six R⁷ groups; provided that when R⁵ is heterocyclyl the heterocyclyl is other than

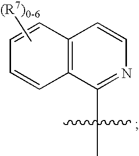

each R⁷ is independently selected from alkoxy, alkoxycarbonyl, alkyl, alkylcarbonyl, aryl, carboxy, cyano, cyanoalkyl, cycloalkyl, halo, haloalkyl, haloalkoxy, heterocyclyl, hydroxy, hydroxyalkyl, nitro, NR^{c}R^{d}, (NR^{c}R^{d})alkyl, (NR^{c}R^{d})alkoxy, (NR^{e}R^{f})carbonyl, and (NR^{e}R^{f})sulfonyl; or

two adjacent R⁷ groups, together with the carbon atoms to which they are attached, form a four- to seven-membered partially- or fully-unsaturated ring optionally containing one or two heteroatoms independently selected from nitrogen, oxygen, and sulfur, wherein the ring is optionally substituted with one, two, or three groups independently selected from alkoxy, alkyl, cyano, halo, haloalkoxy, and haloalkyl;

R^{a} and R^{b} are independently selected from hydrogen, alkoxy, alkyl, aryl, arylalkyl, cycloalkyl, (cycloalkyl)alkyl, heterocyclyl, and heterocyclylalkyl; or R^{a} and R^{b} together with the nitrogen atom to which they are attached form a four- to seven-membered monocyclic heterocyclic ring;

R^{c} and R^{d} are independently selected from hydrogen, alkoxyalkyl, alkoxycarbonyl, alkyl, alkylcarbonyl, arylalkyl, and haloalkyl;

R^{e} and R^{f} are independently selected from hydrogen, alkyl, aryl, arylalkyl, and heterocyclyl; wherein the aryl, the aryl part of the arylalkyl, and the heterocyclyl are optionally substituted with one or two substituents independently selected from alkoxy, alkyl, and halo; and

R^{g} and R^{h} are independently selected from hydrogen, alkyl, aryl, arylalkyl, cycloalkyl, (cycloalkyl)alkyl, heterocyclyl, and heterocyclyl; or R^{g} and R^{h} together with the nitrogen atom to which they are attached form a monocyclic heterocyclic ring wherein the monocyclic heterocyclic ring is optionally fused to a phenyl ring to form a bicyclic system; wherein the monocyclic heterocyclic ring and the bicyclic system are optionally substituted with one, two, or three substituents independently selected from alkoxy, alkyl, halo, haloalkoxy, and haloalkyl.

In a fourth embodiment of the first aspect the present disclosure provides a compound of Formula (I), or a pharmaceutically acceptable salt thereof, wherein

m is 1;

R¹ is —NHSO₂R⁶; wherein R⁶ is unsubstituted cycloalkyl;

R² is alkenyl;

R³ is alkyl;

R⁴ is selected from phenyl and a five- or six-membered partially or fully unsaturated ring optionally containing one, two, three, or four heteroatoms selected from nitrogen, oxygen, and sulfur; wherein each of the rings is optionally substituted with one, two, or three substitutents independently selected from alkoxy, alkoxycarbonyl, alkyl, alkylcarbonyl, carboxy, cyano, cycloalkyl, cycloalkyloxy, halo, haloalkyl, haloalkoxy, —NR^{c}R^{d}, (NR^{e}R^{f})carbonyl, (NR^{e}R^{f})sulfonyl, and oxo; provided that when R⁴ is a six-membered substituted ring all substituents on the ring other than fluoro must be in the meta and/or para positions relative to the ring's point of attachment to the parent molecular moiety;

R⁵ is selected from heterocyclyl and (NR^{g}R^{h})carbonyl, wherein the heterocycyl is optionally substituted with from one to six R⁶ groups; provided that R⁵ is other than

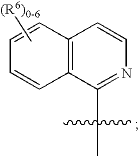

each R⁶ is independently selected from alkoxy, aryl, and heterocyclyl;

R^{c} and R^{d} are independently selected from hydrogen, alkoxycarbonyl, alkyl, alkylcarbonyl, and arylalkyl;

R^{e} and R^{f} are independently selected from hydrogen, alkyl, aryl, and arylalkyl; and

R^{g} and R^{h} together with the nitrogen atom to which they are attached form a monocyclic heterocyclic ring fused to a phenyl ring to form a bicyclic system; wherein the bicyclic system is substituted with a halo group.

In a fifth embodiment of the first aspect the present disclosure provides a compound of Formula (I), or a pharmaceutically acceptable salt thereof, wherein

m is 1;

R¹ is —NHSO₂R⁶; wherein R⁶ is unsubstituted cycloalkyl;

R² is alkenyl;

R³ is alkyl;

R⁴ is six-membered unsaturated ring containing one nitrogen atom wherein the ring is optionally substituted with one, two, or three substitutents independently selected from alkoxy, alkoxycarbonyl, alkyl, alkylcarbonyl, carboxy, cyano, cycloalkyl, cycloalkyloxy, halo, haloalkyl, haloalkoxy, —NR^{c}R^{d}, (NR^{e}R^{f})carbonyl, (NR^{e}R^{f})sulfonyl, and oxo; provided that all substituents on the ring other than fluoro must be in the meta and/or para positions relative to the ring's point of attachment to the parent molecular moiety;

R⁵ is selected from heterocyclyl and (NR^{g}R^{h})carbonyl, wherein the heterocycyl is optionally substituted with from one to six R⁶ groups; provided that R⁵ is other than

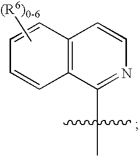

each R⁶ is independently selected from alkoxy, aryl, and heterocyclyl;

R^{c} and R^{d} are independently selected from hydrogen, alkoxycarbonyl, alkyl, alkylcarbonyl, and arylalkyl;

R^{e} and R^{f} are independently selected from hydrogen, alkyl, aryl, and arylalkyl; and

R^{g} and R^{h} together with the nitrogen atom to which they are attached form a monocyclic heterocyclic ring fused to a phenyl ring to form a bicyclic system; wherein the bicyclic system is substituted with a halo group.

In a sixth embodiment of the first aspect the present disclosure provides a compound of Formula (I), or a pharmaceutically acceptable salt thereof, wherein

m is 1;

R¹ is —NHSO₂R⁶; wherein R⁶ is unsubstituted cycloalkyl;

R² is alkenyl;

R³ is alkyl;

R⁴ is five-membered unsaturated ring containing one nitrogen atom and one sulfur atom, wherein the ring is optionally substituted with one, two, or three substitutents independently selected from alkoxy, alkoxycarbonyl, alkyl, alkylcarbonyl, carboxy, cyano, cycloalkyl, cycloalkyloxy, halo, haloalkyl, haloalkoxy, —NR^{c}R^{d}, (NR^{e}R^{f})carbonyl (NR^{e}R^{f})sulfonyl and oxo;

R⁵ is selected from heterocyclyl and (NR^{g}R^{h})carbonyl, wherein the heterocycyl is optionally substituted with from one to six R⁶ groups; provided that R⁵ is other than

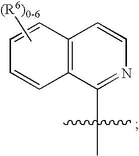

each R⁶ is independently selected from alkoxy, aryl, and heterocyclyl;

R^{c} and R^{d} are independently selected from hydrogen, alkoxycarbonyl, alkyl, alkylcarbonyl, and arylalkyl;

R^{e} and R^{f} are independently selected from hydrogen, alkyl, aryl, and arylalkyl; and

R^{g} and R^{h} together with the nitrogen atom to which they are attached form a monocyclic heterocyclic ring fused to a phenyl ring to form a bicyclic system; wherein the bicyclic system is substituted with a halo group.

In a second aspect the present disclosure provides a composition comprising a compound of formula (I), or a pharmaceutically acceptable salt thereof, and a pharmaceutically acceptable carrier. In a first embodiment of the second aspect the composition further comprises at least one additional compound having anti-HCV activity. In a second embodiment of the second aspect at least one of the additional compounds is an interferon or a ribavirin. In a third embodiment of the second aspect the interferon is selected from interferon alpha 2B, pegylated interferon alpha, consensus interferon, interferon alpha 2A, and lymphoblastiod interferon tau.

In a fourth embodiment of the second aspect the present disclosure provides a composition comprising a compound of formula (I), or a pharmaceutically acceptable salt thereof, a pharmaceutically acceptable carrier, and at least one additional compound having anti-HCV activity; wherein at least one of the additional compounds is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimanitadine.

In a fifth embodiment of the second aspect the present disclosure provides a composition comprising a compound of formula (I), or a pharmaceutically acceptable salt thereof a pharmaceutically acceptable carrier, and at least one additional compound having anti-HCV activity; wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, HCV NS5A protein, and IMPDH for the treatment of an HCV infection.

In a third aspect the present disclosure provides a method of treating an HCV infection in a patient, comprising administering to the patient a therapeutically effective amount of a compound of formula (I), or a pharmaceutically acceptable salt thereof. In a first embodiment of the third aspect the method further comprises administering at least one additional compound having anti-HCV activity prior to, after, or simultaneously with the compound of formula (I), or a pharmaceutically acceptable salt thereof. In a second embodiment of the third aspect at least one of the additional compounds is an interferon or a ribavirin. In a fourth embodiment of the third aspect the interferon is selected from interferon alpha 2B, pegylated interferon alpha, consensus interferon, interferon alpha 2A, and lymphoblastiod interferon tau.

In a fifth embodiment of the third aspect the present disclosure provides a method of treating an HCV infection in a patient, comprising administering to the patient a therapeutically effective amount of a compound of formula (I), or a pharmaceutically acceptable salt thereof; and at least one additional compound having anti-HCV activity prior to, after, or simultaneously with the compound of formula (I), or a pharmaceutically acceptable salt thereof; wherein at least one of the additional compounds is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

In a sixth embodiment of the third aspect the present disclosure provides a method of treating an HCV infection in a patient, comprising administering to the patient a therapeutically effective amount of a compound of formula (I), or a pharmaceutically acceptable salt thereof, and at least one additional compound having anti-HCV activity prior to, after, or simultaneously with the compound of formula (I), or a pharmaceutically acceptable salt thereof, wherein at least one of the additional compounds is effective to inhibit the function of a target selected from HCV metalloprotease, HCV serine protease, HCV polymerase, HCV helicase, HCV NS4B protein, HCV entry, HCV assembly, NCV egress, HCV NS5A protein, and IMPDH for the treatment of an HCV infection.

In a fourth aspect the present disclosure provides a composition comprising a compound of formula (I), or a pharmaceutically acceptable salt thereof; one, two, three, four, or five additional compounds having anti-HCV activity, and a pharmaceutically acceptable carrier. In a first embodiment of the fourth aspect the composition comprises three or four additional compounds having anti-HCV activity. In a second embodiment of the fourth aspect the composition comprises one or two additional compounds having anti-HCV activity.

In a fifth aspect the present disclosure provides a method of treating an HCV infection in a patient, comprising administering to the patient a therapeutically effective amount of a compound of formula (I), or a pharmaceutically acceptable salt thereof and one, two, three, four, or five additional compounds having anti-HCV activity prior to, after, or simultaneously with the compound of formula (I), or a pharmaceutically acceptable salt thereof. In a first embodiment of the first aspect the method comprises administering three or four additional compounds having anti-HCV activity. In a second embodiment of the first aspect the method comprises administering one or two additional compounds having anti-HCV activity.

Other aspects of the present disclosure may include suitable combinations of embodiments disclosed herein.

Yet other aspects and embodiments may be found in the description provided herein.

The description of the present disclosure herein should be construed in congruity with the laws and principals of chemical bonding. In some instances it may be necessary to remove a hydrogen atom in order accommodate a substitutent at any given location.

It should be understood that the compounds encompassed by the present disclosure are those that are suitably stable for use as pharmaceutical agent.

It is intended that the definition of any substituent or variable at a particular location in a molecule be independent of its definitions elsewhere in that molecule.

All patents, patent applications, and literature references cited in the specification are herein incorporated by reference in their entirety. In the case of inconsistencies, the present disclosure, including definitions, will prevail.

As used in the present specification, the following terms have the meanings indicated:

As used herein, the singular forms “a”, “an”, and “the” include plural reference unless the context clearly dictates otherwise.

Unless stated otherwise, all aryl, cycloalkyl, and heterocyclyl groups of the present disclosure may be substituted as described in each of their respective definitions. For example, the aryl part of an arylalkyl group may be substituted as described in the definition of the term ‘aryl’.

In some instances, the number of carbon atoms in any particular group is denoted before the recitation of the group. For example, the term “C₆ alkyl” denotes an alkyl group containing six carbon atoms. Where these designations exist they supercede all other definitions contained herein.

As used herein, the singular forms “a”, “an”, and “the” include plural reference unless the context clearly dictates otherwise.

The term “alkenyl,” as used herein, refers to a straight or branched chain group of two to six carbon atoms containing at least one carbon-carbon double bond.

The term “alkoxy,” as used herein, refers to an alkyl group attached to the parent molecular moiety through an oxygen atom.

The term “alkoxyalkyl,” as used herein, refers to an alkyl group substituted with one, two, or three alkoxy groups.

The term “alkoxycarbonyl,” as used herein, refers to an alkoxy group attached to the parent molecular moiety through a carbonyl group.

The term “alkoxycarbonylalkyl,” as used herein, refers to an alkyl group substituted with one, two, or three alkoxycarbonyl groups.

The term “alkyl,” as used herein, refers to a group derived from a straight or branched chain saturated hydrocarbon containing from one to ten carbon atoms.

The term “alkylcarbonyl,” as used herein, refers to an alkyl group attached to the parent molecular moiety through a carbonyl group.

The term “alkylsulfanyl,” as used herein, refers to an alkyl group attached to the parent molecular moiety through a sulfur atom.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl. The aryl groups of the present disclosure can be optionally substituted with one, two, three, four, or five substituents independently selected from alkoxy, alkoxycarbonyl, alkyl, alkylcarbonyl, carboxy, cycloalkyl, cycloalkyloxy, cyano, halo, haloalkoxy, haloalkyl, nitro, —NR^{c}R^{d}, (NR^{c}R^{d})carbonyl, and oxo.

The term “arylalkyl,” as used herein, refers to an alkyl group substituted with one, two, or three aryl groups.

The term “arylalkylcarbonyl,” as used herein, refers to an arylalkyl group attached to the parent molecular moeity through a carbonyl group.

The term “arylcarbonyl,” as used herein, refers to an aryl group attached to the parent molecular moiety through a carbonyl group.

The term “carbonyl,” as used herein, refers to —C(O)—.

The term “carboxy,” as used herein, refers to —CO₂H.

The term “carboxyalkyl,” as used herein, refers to an alkyl group substituted with one, two, or three carboxy groups.

The term “cyano,” as used herein, refers to —CN,

The term “cyanoalkyl,” as used herein, refers to an alkyl group substituted with one, two, or three cyano groups.

The term “cycloalkenyl,” as used herein, refers to a non-aromatic, partially unsaturated monocyclic, bicyclic, or tricyclic ring system having three to fourteen carbon atoms and zero heteroatoms. Representative examples of cycloalkenyl groups include, but are not limited to, cyclohexenyl, octahydronaphthalenyl, and norbornylenyl.

The term “cycloalkyl,” as used herein, refers to a saturated monocyclic or bicyclic hydrocarbon ring system having three to ten carbon atoms and zero heteroatoms. Representative examples of cycloalkyl groups include, but are not limited to, cyclopropyl, cyclobutyl, and cyclopentyl.

The term “(cycloalkyl)alkyl,” as used herein, refers to an alkyl group substituted with one, two, or three cycloalkyl groups.

The term “cycloalkyloxy,” as used herein, refers to a cycloalkyl group attached to the parent molecular moiety through an oxygen atom.

The terms “halo” and “halogen,” as used herein, refer to F, Cl, Br, and I.

The term “haloalkoxy,” as used herein, refers to a haloalkyl group attached to the parent molecular moiety through an oxygen atom.

The term “haloalkyl,” as used herein, refers to an alkyl group substituted with one, two, three, or four halogen atoms.

The term “heterocyclyl,” as used herein, refers to a five-, six-, or seven-membered ring containing one, two, or three heteroatoms independently selected from nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a four- to six-membered aromatic or non-aromatic carbocyclic ring or another monocyclic heterocyclyl group. The heterocyclyl groups of the present disclosure can be attached to the parent molecular moiety through a carbon atom or a nitrogen atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl. The heterocyclyl groups of the present disclosure can be optionally substituted with one, two, three, four, or five substituents independently selected from alkoxy, alkoxycarbonyl, alkyl, alkylcarbonyl, carboxy, cycloalkyl, cycloalkyloxy, cyano, halo, haloalkoxy, haloalkyl, nitro, —NR^{c}R^{d}, (NR^{c}R^{d})carbonyl, and oxo.

The term “heterocyclylalkyl,” as used herein, refers to an alkyl group substituted with one, two, or three heterocyclyl groups.

The term “heterocyclylalkylcarbonyl,” as used herein, refers to a heterocyclylalkyl group attached to the parent molecular moiety through a carbonyl group.

The term “heterocyclylcarbonyl,” as used herein, refers to a heterocyclyl group attached to the parent molecular moiety through a carbonyl group.

The term “hydroxy,” as used herein, refers to —OH.

The term “hydroxyalkyl,” as used herein, refers to an alkyl group substituted with one, two, or three hydroxy groups.

The term “nitro,” as used herein, refers to —NO₂.

The term “—NR^{a} R^{b},” as used herein, refers to two groups, R^{a} and R^{b}, which are attached to the parent molecular moiety through a nitrogen atom. R^{a} and R^{b} are independently selected from hydrogen, alkoxy, alkyl, aryl, arylalkyl, cycloalkyl, (cycloalkyl)alkyl, heterocyclyl, and heterocyclylalkyl; or R^{a} and R^{b} together with the nitrogen atom to which they are attached form a five or six-membered monocyclic heterocyclic ring.

The term “—NR^{c}R^{d},” as used herein, refers to two groups, R^{c} and R^{d}, which are attached to the parent molecular moiety through a nitrogen atom. R^{c} and R^{d} are independently selected from hydrogen, alkoxycarbonyl, alkyl, and alkylcarbonyl.

The term “(NR^{c}R^{d})alkoxy,” as used herein, refers to an (NR^{c}R^{d})alkyl group attached to the parent molecular moiety through an oxygen atom.

The term “(NR^{c}R^{d})alkyl,” as used herein, refers to an alkyl group substituted with one, two, or three —NR^{c}R^{d} groups.

The term (NR^{c}R^{d})carbonyl,” as used herein, refers to an —NR^{c}R^{d} group attached to the parent molecular moiety through a carbonyl group.

The term “—NR^{e}R^{f},” as used herein, refers to two groups, R^{e} and R^{f}, which are attached to the parent molecular moiety through a nitrogen atom. R^{e} and R^{f} are independently selected from hydrogen, alkyl, aryl, and arylalkyl.

The term “(NR^{e}R^{f})carbonyl,” as used herein, refers to an —NR^{e}R^{f} group attached to the parent molecular moiety through a carbonyl group.

The term “(NR^{e}R^{f})carbonylalkyl,” as used herein, refers to an (NR^{e}R^{f})carbonyl group attached to the parent molecular moiety through an alkyl group.

The term “(NR^{e}R^{f})sulfonyl,” as used herein, refers to an —NR^{c}R^{f} group attached to the parent molecular moiety through a sulfonyl group.

The term “(NR^{g}R^{h})carbonyl,” as used herein, refers to an —NR^{g}R^{h} group attached to the parent molecular moiety through a carbonyl group.

The term “—NR^{g}R^{h},” as used herein, refers to two groups, R^{g} and R^{h}, which are attached to the parent molecular moiety through a nitrogen atom. R^{g} and R^{h} are independently selected from hydrogen, alkyl, aryl, arylalkyl, cycloalkyl, (cycloalkyl)alkyl, heterocyclyl, and heterocyclyl; or R^{g} and R^{h} together with the nitrogen atom to which they are attached form a monocyclic heterocyclic ring wherein the monocyclic heterocyclic ring is optionally fused to a phenyl ring to form a bicyclic system; wherein the monocyclic heterocyclic ring and the bicyclic system are optionally substituted with one, two, or three substituents independently selected from alkoxy, alkyl, halo, haloalkoxy, and haloalkyl.

The term “oxo,” as used herein, refers to ═O.

The term “sulfonyl,” as used herein, refers to —SO₂—.

The term “prodrug,” as used herein, represents compounds which are rapidly transformed in vivo to the parent compounds by hydrolysis in blood. Prodrugs of the present disclosure include esters of hydroxy groups on the parent molecule, esters of carboxy groups on the parent molecule, and amides of the amines on the parent molecule.

The compounds of the present disclosure can exist as pharmaceutically acceptable salts. The term “pharmaceutically acceptable salt,” as used herein, represents salts or zwitterionic forms of the compounds of the present disclosure which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use. The salts can be prepared during the final isolation and purification of the compounds or separately by reacting a suitable basic functionality with a suitable acid. Representative acid addition salts include acetate, adipate, alginate, citrate, aspartate, benzoate, benzenesulfonate, bisulfate, butyrate, camphorate, camphorsulfonate; digluconate, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds by reacting an acidic group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylpheniethylamine, and N,N′-dibenzylethylenediamine. Other representative organic amines useful for the formation of base addition salts include ethylenediamine, ethanolamine, diethanolamine, piperidine, and piperazine.

As used herein, the term “anti-HCV activity” means the compound is effective to treat the HCV virus.

The term “compounds of the disclosure”, and equivalent expressions, are meant to embrace compounds of formula (I), and pharmaceutically acceptable enantiomers, diastereomers, and salts thereof. Similarly, references to intermediates, are meant to embrace their salts where the context so permits.

The term “patient” includes both human and other mammals.

The term “pharmaceutical composition” means a composition comprising a compound of the disclosure in combination with at least one additional pharmaceutical carrier, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Ingredients listed in Remington's Pharmaceutical Sciences, 18^{th} ed., Mack Publishing Company, Easton, Pa. (1999) for example, may be used.

The phrase “pharmaceutically acceptable” is employed herein to refer to those compounds, materials, compositions, and/or dosage forms which are, within the scope of sound medical judgment, suitable for use in contact with the tissues of patients without excessive toxicity, irritation, allergic response, or other problem or complication commensurate with a reasonable risk/benefit ratio.

The term “therapeutically effective amount” means the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “treat” and “treating” refers to: (i) preventing a disease, disorder or condition from occurring in a patient which may be predisposed to the disease, disorder and/or condition but has not yet been diagnosed as having it; (ii) inhibiting the disease, disorder or condition, i.e., arresting its development; and/or (iii) relieving the disease, disorder or condition, i.e., causing regression of the disease, disorder and/or condition.

Where used in naming compounds of the present disclosure, the designations P1′, P1, P2, P2*, P3, and P4, as used herein, map the relative positions of the amino acid residues of a protease inhibitor binding relative to the binding of the natural peptide cleavage substrate. Cleavage occurs in the natural substrate between P1and P1′ where the nonprime positions designate amino acids starting from the C-terminus end of the peptide natural cleavage site extending towards the N-terminus; whereas, the prime positions emanate from the N-terminus end of the cleavage site designation and extend toward the C-terminus. For example, P1′ refers to the first position away from the right hand end of the C-terminus of the cleavage site (i.e. N-terminus first position); whereas P1 starts the numbering from the left hand side of the C-terminus cleavage site, P2: second position from the C-terminus, etc.). (see Berger A. & Schechter I., Transactions of the Royal Society London series (1970), B257, 249-264].

The following figure shows the designations for the compounds of the present disclosure.

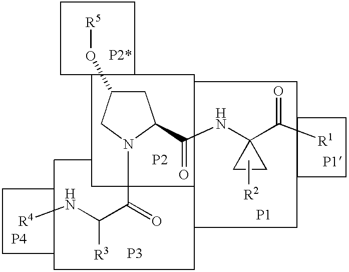

Asymmetric centers exist in the compounds of the present disclosure. For example, the compounds may include P1 cyclopropyl element of formula

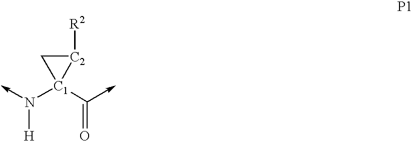

wherein C₁ and C₂ each represent an asymmetric carbon atom at positions 1 and 2 of the cyclopropyl ring.

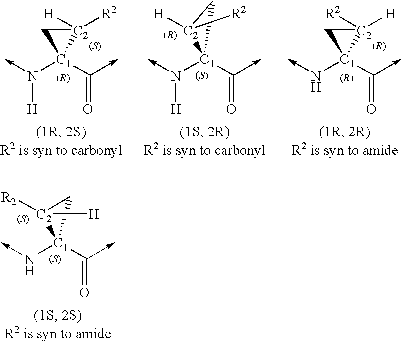

It should be understood that the disclosure encompasses all stereochemical forms, or mixtures thereof, which possess the ability to inhibit HCV protease.

Certain compounds of the present disclosure may also exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Certain compounds of the present disclosure may exist in zwitterionic form and the present disclosure includes each zwitterionic form of these compounds and mixtures thereof.

When it is possible that, for use in therapy, therapeutically effective amounts of a compound of formula (I), as well as pharmaceutically acceptable salts thereof, may be administered as the raw chemical, it is possible to present the active ingredient as a pharmaceutical composition. Accordingly, the disclosure further provides pharmaceutical compositions, which include therapeutically effective amounts of compounds of formula (I) or pharmaceutically acceptable salts thereof, and one or more pharmaceutically acceptable carriers, diluents, or excipients. The compounds of formula (I) and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the disclosure there is also provided a process for the preparation of a pharmaceutical formulation including admixing a compound of formula (I), or a pharmaceutically acceptable salt thereof, with one or more pharmaceutically acceptable carriers, diluents, or excipients.

Pharmaceutical formulations may be presented in unit dose forms containing a predetermined amount of active ingredient per unit dose. Dosage levels of between about 0.01 and about 250 milligram per kilogram (“mg/kg”) body weight per day, preferably between about 0.05 and about 100 mg/kg body weight per day of the compounds of the disclosure are typical in a monotherapy for the prevention and treatment of HCV mediated disease. Typically, the pharmaceutical compositions of this disclosure will be administered from about 1 to about 5 times per day or alternatively, as a continuous infusion. Such administration can be used as a chronic or acute therapy. The amount of active ingredient that may be combined with the carrier materials to produce a single dosage form will vary depending on the condition being treated, the severity of the condition, the time of administration, the route of administration, the rate of excretion of the compound employed, the duration of treatment, and the age, gender, weight, and condition of the patient. Preferred unit dosage formulations are those containing a daily dose or sub-dose, as herein above recited, or an appropriate fraction thereof, of an active ingredient. Generally, treatment is initiated with small dosages substantially less than the optimum dose of the compound. Thereafter, the dosage is increased by small increments until the optimum effect under the circumstances is reached. In general, the compound is most desirably administered at a concentration level that will generally afford antivirally effective results without causing any harmful or deleterious side effects.

When the compositions of this disclosure comprise a combination of a compound of the disclosure and one or more additional therapeutic or prophylactic agent, both the compound and the additional agent are usually present at dosage levels of between about 10 to 150%, and more preferably between about 10 and 80% of the dosage normally administered in a monotherapy regimen.

Pharmaceutical formulations may be adapted for administration by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present disclosure can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided, Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The compounds of formula (I), and pharmaceutically acceptable salts thereof, can also be administered in the form of liposome delivery systems, such as small unilamellar vesicles, large unilamellar vesicles, and multilamellar vesicles. Liposomes can be formed from a variety of phopholipids, such as cholesterol, stearylamine, or phophatidylcholines.

The compounds of formula (I) and pharmaceutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouth washes.

Pharmaceutical formulations adapted for rectal administration may be presented as suppositories or as enemas.

Pharmaceutical formulations adapted for nasal administration wherein the carrier is a solid include a course powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insuflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and soutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

Table 1 below lists some illustrative examples of compounds that can be administered with the compounds of this disclosure. The compounds of the disclosure can be administered with other anti-HCV activity compounds in combination therapy, either jointly or separately, or by combining the compounds into a composition.

TABLE 1

| | | Type of Inhibitor or | |
|---|---|---|---|
| Brand Name | Physiological Class | Target | Source Company |
| NIM811 | | Cyclophilin Inhibitor | Novartis |
| Zadaxin | | Immuno-modulator | Sciclone |
| Suvus | | Methylene blue | Bioenvision |
| Actilon | | TLR9 agonist | Coley |
| (CPG10101) | | | |
| Batabulin (T67) | Anticancer | β-tubulin inhibitor | Tularik Inc., |
| | | | South San |
| | | | Francisco, CA |
| ISIS 14803 | Antiviral | antisense | ISIS |
| | | | Pharmaceuticals |
| | | | Inc, Carlsbad, |
| | | | CA/Elan |
| | | | Phamaceuticals |
| | | | Inc., New York, |
| | | | NY |
| Summetrel | Antiviral | antiviral | Endo |
| | | | Pharmaceuticals |
| | | | Holdings Inc., |
| | | | Chadds Ford, PA |
| GS-9132 (ACH- | Antiviral | HCV Inhibitor | Achillion/ |
| 806) | | | Gilead |
| Pyrazolopyrimidine | Antiviral | HCV Inhibitors | Arrow |
| compounds and | | | Therapeutics Ltd. |
| salts | | | |
| From WO- | | | |
| 2005047288 | | | |
| 26 May 2005 | | | |
| Levovirin | Antiviral | IMPDH inhibitor | Ribapharm Inc., |
| | | | Costa Mesa, CA |
| Merimepodib | Antiviral | IMPDH inhibitor | Vertex |
| (VX-497) | | | Pharmaceuticals |
| | | | Inc., Cambridge, |
| | | | MA |
| XTL-6865 (XTL- | Antiviral | monoclonal antibody | XTL |
| 002) | | | Biopharmaceuticals |
| | | | Ltd., Rehovot, |
| | | | Isreal |
| Telaprevir | Antiviral | NS3 serine protease | Vertex |
| (VX-950, LY- | | inhibitor | Pharmaceuticals |
| 570310) | | | Inc., Cambridge, |
| | | | MA/Eli Lilly |
| | | | and Co. Inc., |
| | | | Indianapolis, IN |
| HCV-796 | Antiviral | NS5B Replicase | Wyeth/ |
| | | Inhibitor | Viropharma |
| NM-283 | Antiviral | NS5B Replicase | Idenix/Novartis |
| | | Inhibitor | |
| GL-59728 | Antiviral | NS5B Replicase | Gene Labs/ |
| | | Inhibitor | Novartis |
| GL-60667 | Antiviral | NS5B Replicase | Gene Labs/ |
| | | Inhibitor | Novartis |
| 2′C MeA | Antiviral | NS5B Replicase | Gilead |
| | | Inhibitor | |
| PSI 6130 | Antiviral | NS5B Replicase | Roche |
| | | Inhibitor | |
| R1626 | Antiviral | NS5B Replicase | Roche |
| | | Inhibitor | |
| 2′C Methyl | Antiviral | NS5B Replicase | Merck |
| adenosine | | Inhibitor | |
| JTK-003 | Antiviral | RdRp inhibitor | Japan Tobacco |
| | | | Inc., Tokyo, |
| | | | Japan |
| Levovirin | Antiviral | ribavirin | ICN |
| | | | Pharmaceuticals, |
| | | | Costa Mesa, CA |
| Ribavirin | Antiviral | ribavirin | Schering-Plough |
| | | | Corporation, |
| | | | Kenilworth, NJ |
| Viramidine | Antiviral | Ribavirin Prodrug | Ribapharm Inc., |
| | | | Costa Mesa, CA |
| Heptazyme | Antiviral | ribozyme | Ribozyme |
| | | | Pharmaceuticals |
| | | | Inc., Boulder, CO |
| BILN-2061 | Antiviral | serine protease | Boehringer |
| | | inhibitor | Ingelheim |
| | | | Pharma KG, |
| | | | Ingelheim, |
| | | | Germany |
| SCH 503034 | Antiviral | serine protease | Schering Plough |
| | | inhibitor | |
| Zadazim | Immune modulator | Immune modulator | SciClone |
| | | | Pharmaceuticals |
| | | | Inc., San Mateo, |
| | | | CA |
| Ceplene | Immunomodulator | immune modulator | Maxim |
| | | | Pharmaceuticals |
| | | | Inc., San Diego, |
| | | | CA |
| CellCept | Immunosuppressant | HCV IgG immuno- | F. Hoffmann-La |
| | | suppressant | Roche LTD, |
| | | | Basel, |
| | | | Switzerland |
| Civacir | Immunosuppressant | HCV IgG immuno- | Nabi |
| | | suppressant | Biopharmaceuticals |
| | | | Inc., Boca |
| | | | Raton, FL |
| Albuferon-α | Interferon | albumin IFN-α2b | Human Genome |
| | | | Sciences Inc., |
| | | | Rockville, MD |
| Infergen A | Interferon | IFN | InterMune |
| | | alfacon-1 | Pharmaceuticals |
| | | | Inc., Brisbane, |
| | | | CA |
| Omega IFN | Interferon | IFN-ω | Intarcia |
| | | | Therapeutics |
| IFN-β and EMZ701 | Interferon | IFN-β and EMZ701 | Transition |
| | | | Therapeutics |
| | | | Inc., Ontario, |
| | | | Canada |
| Rebif | Interferon | IFN-β1a | Serono, Geneva, |
| | | | Switzerland |
| Roferon A | Interferon | IFN-α2a | F. Hoffmann-La |
| | | | Roche LTD, |
| | | | Basel, |
| | | | Switzerland |
| Intron A | Interferon | IFN-α2b | Schering-Plough |
| | | | Corporation, |
| | | | Kenilworth, NJ |
| Intron A and | Interferon | IFN-α2b/α1-thymosin | RegeneRx |
| Zadaxin | | | Biopharma. Inc., |
| | | | Bethesda, MD/ |
| | | | SciClone |
| | | | Pharmaceuticals |
| | | | Inc, San Mateo, |
| | | | CA |
| Rebetron | Interferon | IFN-α2b/ribavirin | Schering-Plough |
| | | | Corporation, |
| | | | Kenilworth, NJ |
| Actimmune | Interferon | INF-γ | InterMune Inc., |
| | | | Brisbane, CA |
| Interferon-β | Interferon | Interferon-β-1a | Serono |
| Multiferon | Interferon | Long lasting IFN | Viragen/ |
| | | | Valentis |
| Wellferon | Interferon | Lympho-blastoid IFN- | GlaxoSmithKline |
| | | αn1 | plc, Uxbridge, |
| | | | UK |
| Omniferon | Interferon | natural IFN-α | Viragen Inc., |
| | | | Plantation, FL |
| Pegasys | Interferon | PEGylated IFN-α2a | F. Hoffmann-La |
| | | | Roche LTD, |
| | | | Basel, |
| | | | Switzerland |
| Pegasys and | Interferon | PEGylated IFN-α2a/ | Maxim |
| Ceplene | | immune modulator | Pharmaceuticals |
| | | | Inc., San Diego, |
| | | | CA |
| Pegasys and | Interferon | PEGylated IFN- | F. Hoffmann-La |
| Ribavirin | | α2a/ribavirin | Roche LTD, |
| | | | Basel, |
| | | | Switzerland |
| PEG-Intron | Interferon | PEGylated IFN-α2b | Schering-Plough |
| | | | Corporation, |
| | | | Kenilworth, NJ |
| PEG-Intron/ | Interferon | PEGylated IFN- | Schering-Plough |
| Ribavirin | | α2b/ribavirin | Corporation, |
| | | | Kenilworth, NJ |
| IP-501 | Liver protection | antifibrotic | Indevus |
| | | | Pharmaceuticals |
| | | | Inc., Lexington, |
| | | | MA |
| IDN-6556 | Liver protection | caspase inhibitor | Idun |
| | | | Pharmaceuticals |
| | | | Inc., San Diego, |
| | | | CA |
| ITMN-191 (R-7227) | Antiviral | serine protease | InterMune |
| | | inhibitor | Pharmaceuticals |
| | | | Inc., Brisbane, |
| | | | CA |
| GL-59728 | Antiviral | NS5B Replicase | Genelabs |
| | | Inhibitor | |
| ANA-971 | Antiviral | TLR-7 agonist | Anadys |
| Boceprevir | Antiviral | serine protease | Schering Plough |
| | | inhibitor | |
| TMS-435 | Antiviral | serine protease | Tibotec BVBA, |
| | | inhibitor | Mechelen, |
| | | | Belgium |
| BI-201335 | Antiviral | serine protease | Boehringer |
| | | inhibitor | Ingelheim |
| | | | Pharma KG, |
| | | | Ingelheim, |
| | | | Germany |
| MK-7009 | Antiviral | serine protease | Merck |
| | | inhibitor | |
| PF-00868554 | Antiviral | replicase inhibitor | Pfizer |
| ANA598 | Antiviral | Non-Nucleoside | Anadys |
| | | NS5B Polymerase | Pharmaceuticals, |
| | | Inhibitor | Inc., San Diego, |
| | | | CA, USA |
| IDX375 | Antiviral | Non-Nucleoside | Idenix |
| | | Replicase Inhibitor | Pharmaceuticals, |
| | | | Cambridge, MA, |
| | | | USA |
| BILB 1941 | Antiviral | NS5B Polymerase | Boehringer |
| | | Inhibitor | Ingelheim |
| | | | Canada Ltd |
| | | | R&D, Laval, QC, |
| | | | Canada |
| PSI-7851 | Antiviral | Nucleoside | Pharmasset, |
| | | Polymerase inhibitor | Princeton, NJ, |
| | | | USA |
| VCH-759 | Antiviral | NS5B Polymerase | ViroChem |
| | | Inhibitor | Pharma |
| VCH-916 | Antiviral | NS5B Polymerase | ViroChem |
| | | Inhibitor | Pharma |
| GS-9190 | Antiviral | NS5B Polymerase | Gilead |
| | | Inhibitor | |
| Peg-interferon | Antiviral | Interferon | ZymoGenetics/Bristol- |
| lamda | | | Myers |
| | | | Squibb |

The compounds of the disclosure may also be used as laboratory reagents. Compounds may be instrumental in providing research tools for designing of viral replication assays, validation of animal assay systems and structural biology studies to further enhance knowledge of the HCV disease mechanisms. Further, the compounds of the present disclosure are useful in establishing or determining the binding site of other antiviral compounds, for example, by competitive inhibition.

The compounds of this disclosure may also be used to treat or prevent viral contamination of materials and therefore reduce the risk of viral infection of laboratory or medical personnel or patients who come in contact with such materials, e.g., blood, tissue, surgical instruments and garments, laboratory instruments and garments, and blood collection or transfusion apparatuses and materials.

This disclosure is intended to encompass compounds having formula (I) when prepared by synthetic processes or by metabolic processes including those occurring in the human or animal body (in vivo) or processes occurring in vitro.

The abbreviations used in the present application, including particularly in the illustrative schemes and examples which follow, are well-known to those skilled in the art. Some of the abbreviations used are as follows: OAc for acetate; t-Bu for tert-butyl; TBMDSCI for tert-butyldimethylsilyl chloride; 1,2-DME for 1,2-dimethoxyethane; DMA for N,N-dimethylacetamide; n-BuLi or n-BuLi for n-butyllithium; THF for tetrahydrofuran; Et₃N for triethylamine; TBME or MTBE for tert-butyl methyl ether; rt or RT for room temperature or retention time (context will dictate); Boc or BOC for tert-butoxycarbonyl; DMSO for dimethylsulfoxide; EtOH for ethanol; MeCN for acetonitrile; TFA for trifluoroacetic acid; h for hours; d for days; EtOAc for ethyl acetate; CDI for 1,1′-carbonyldiimidazole; DBU for 1,8-diazabicyclo[5.4.0]undec-7-ene; DCM for dichloromethane; Et₂O for diethyl ether; HATU for O-(7-azabenzotriazol-1-yl)-N,N,N′,N′-tetramethyluronium phosphate; NMM for N-methylmorpholine; DCE for 1,2-dichloroethane; and DIEA or DIPEA for diisopropylethylamine.

The starting materials useful to synthesize the compounds of the present disclosure are known to those skilled in the art and can be readily manufactured or are commercially available.

The following methods set forth below are provided for illustrative purposes and are not intended to limit the scope of the claims. It will be recognized that it may be necessary to prepare such a compound in which a functional group is protected using a conventional protecting group then to remove the protecting group to provide a compound of the present disclosure. The details concerning the use of protecting groups in accordance with the present disclosure are known to those skilled in the art.

In the construction of compounds of Formula (I) the P1′ terminus is incorporated into the molecules using one of the general processes outlined above and described in more detail below. In some examples the P1′ elements, that is the cycloalkyl or alkyl sulfonamides, are commercially available or can be prepared from the corresponding alkyl- or cycloalkylsulfonyl chloride by treating the sulfonyl chloride with ammonia. Alternatively, these sulfonamides can be synthesized using the general process outlined below. Commercially available 3-chloropropylsulfonyl chloride (1) is converted to a suitably protected sulfonamide, for example, by treatment with tert-butyl amine. The sulfonamide obtained (2) is then converted to the corresponding cycloalkylsulfonamide by treatment with two equivalents of a base such as butyllithium in a solvent such as THF at low temperature. The resulting cycloalkylsulfonamide can be deprotected by treatment with an acid to provide the desired unprotected cycloalkylsulfoamide.

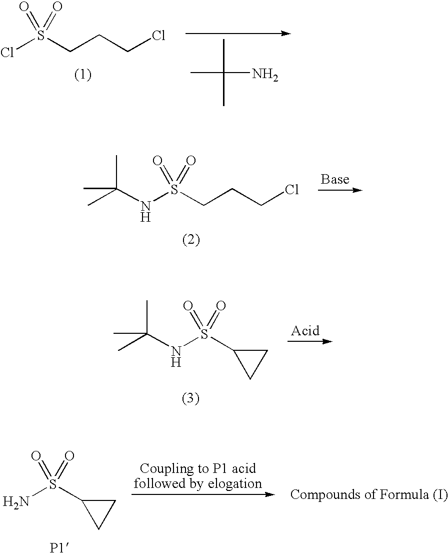

Substituted cycloalkylsulfonamides can also be incorporated into compounds of Formula (I) using a modification of the above said procedure. For example, intermediate 2 shown below can be treated with two equivalents of base such as butyllithium and the resulting reaction mixture can be treated with an electrophile such as methyl iodide to provide a substituted cycloalkylsulfonamide (3). This intermediate (3) can be deprotected at the N-terminus and the resulting compound (4) utilized as an intermediate in the preparation of compounds of Formula (I).

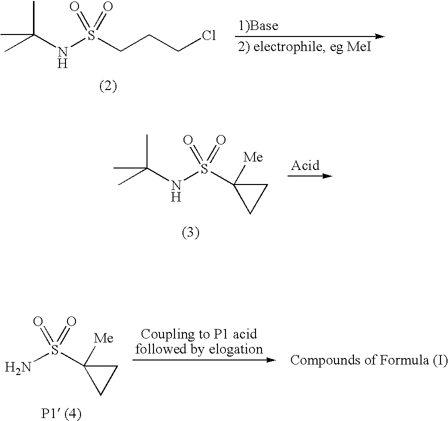

The P1′ intermediates employed in generating compounds of Formula (I) are in some cases derived from sulfamide derivatives. In such cases the sulfamide intermediates are available by several synthetic routes as, for example, by the pathway outlined below.

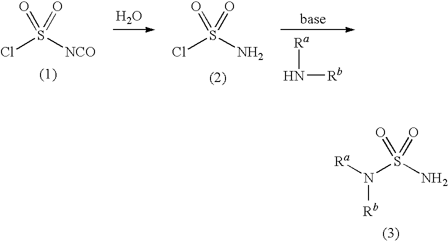

Sulfamoyl chloride (2) can be prepared in situ by the addition of water (e.g., 1 equivalent) to chlorosulfonyl isocyanate 1 (e.g., 1 equivalent) in a solvent such as THF while maintained at a low temperature such as −20° C. The resulting solution is then allowed to warm to 0° C. To this solution a base, such as anhydrous triethylamine (eg., 1 equivalent), is added followed by an amine (eg., 1 equivalent). The reaction mixture is then warmed to room temperature, filtered, and the filtrate concentrated to provide the desired sulfamides (3).

The sulfamides can be incorporated into compounds of Formula (I) by several processes as, for example, by following the synthetic pathway defined in the scheme shown below. A carboxylic acid P1 element (1) is treated with an activating agent such as CDI. In a separate flask, a strong base is added to a solution of the above described sulfamide and the resulting reaction mixture is stirred for several hours after which this reaction mixture is added to the flask containing the activated carboxylic acid, to provide acylsulfamide derivatives (2). Intermediates like 2 can be converted to compounds of Formula (I) as described herein.

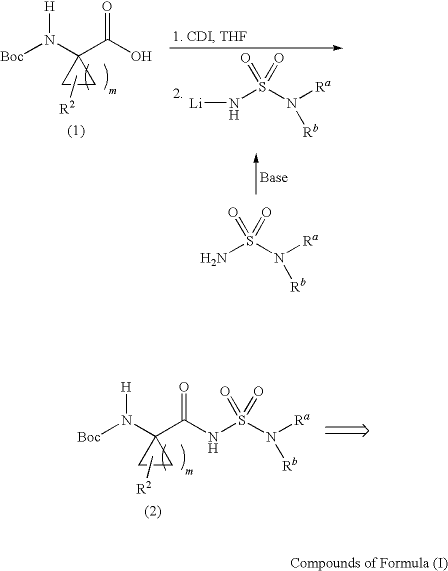

The P1 elements utilized in generating compounds of Formula (I) are in some cases commercially available, but are otherwise synthesized using the methods described herein and are subsequently incorporated into compounds of Formula (I) using the methods described herein. The substituted P1 cyclopropylamino acids can be synthesized following the general process outlined in the scheme below.

Treatment of commercially available or easily synthesized imine (1) with 1,4-dihalobutene (2) in presence of a base provides the resulting imine (3). Acid hydrolysis of 3 then provides 4, which has an allyl substituent syn to the carboxyl group, as a major product. The amine moiety of 4 can protected using a Boc group to provide the fully protected amino acid 5. This intermediate is a racemate which can be resolved by an enzymatic process wherein the ester moiety of 5 is cleaved by a protease to provide the corresponding carboxylic acid. Without being bound to any particular theory, it is believed that this reaction is selective in that one of the enantiomers undergoes the reaction at a much greater rate than its mirror image providing for a kinetic resolution of the intermediate racemate. In the examples cited herein, the more preferred stereoisomer for integration into compounds of Formula (I) is 5a which houses the (1S, 2R) stereochemistry. In the presence of the enzyme, this enantiomer does not undergo ester cleavage and thereby this enantiomer, 5a, is recovered from the reaction mixture. However, the less preferred enantiomer, 5b, which houses the (1S, 2R) stereochemistry, undergoes ester cleavage, i.e., hydrolysis, to provide the free acid 6. Upon completion of this reaction, the ester 5a can be separated from the acid product 6 by routine methods such as, for example, aqueous extraction methods or chromotography.

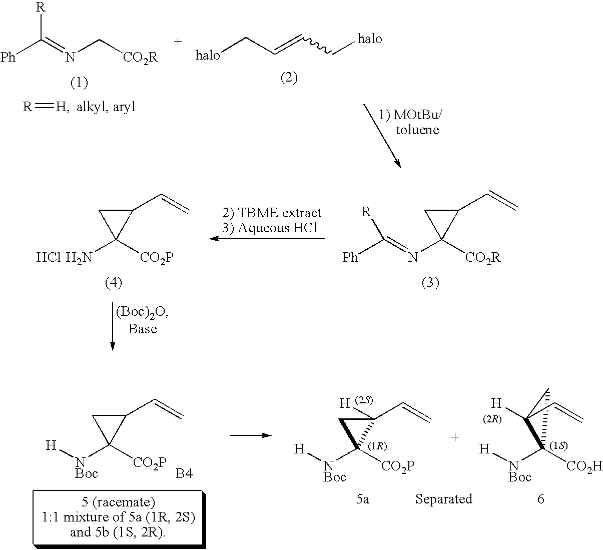

Several of the aminoaryl products were synthesized through traditional peptide coupling of an in-house prepared core dipeptide amine with a commercially available N-arylamino acid fragment. Where the N-arylamino acid fragment was not available commercially, it was synthesized. Synthetic routes to these N-arylamino acid fragments include, but are not limited to, the following:

(1) Nucleophilic aromatic substitution of a sufficiently electrophilic aromatic or heteroaromatic species with an amino acid ester, followed by deesterification of the product:

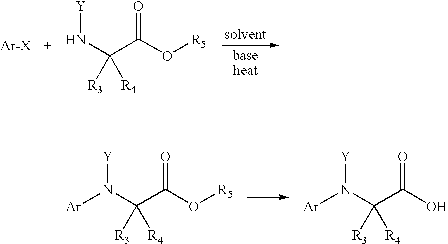

(2) A Buchwald-Hartwig type reaction involving phosphine ligand mediated Pd(0) insertion into an aryl or heteroaryl halide bond followed by displacement with an amino acid tert-butyl ester, followed by deesterification of the product (Shen, Q.; Shekhar, S; Stambuli, J. P.; Hartwig, J. F. Angew. Chem. Int. Ed. 2005, 44, 1371-1375.):

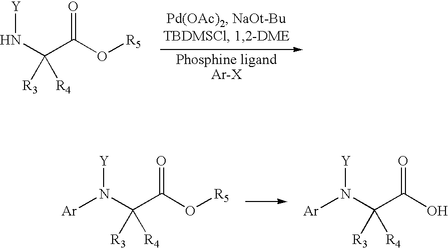

(3) An Ullmann-like condensation whereby an aryl or heteroaryl halide and a free amino acid are made to react via a CuI mediated process to give the arylamino acid directly (Ma, D.; Zhang, Y.; Yao, J.; Wu, S.; Tao, F. J. Am. Chem. Soc. 1998, 120, 12459-12467.):

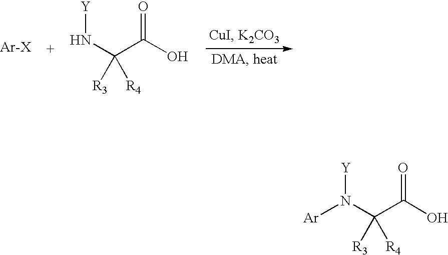

(4) Nucleophilic addition of the dianion of a free amino acid to an aryl or heteroaryl halide, typically a fluoride (similar to Saitton, S.; Kihlberg, J.; Luthman, K. Tetrahedron 2004, 60, 6113-6120.):

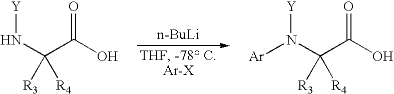

In some cases, access to the aminoaryl final products can be achieved by direct nucleophilic aromatic substitution of the aryl ring with a fully assembled core tripeptide having a free amino group at the terminus of the P3 subregion:

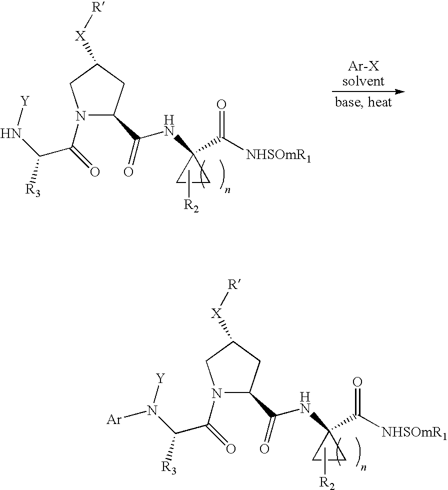

These reactions are limited to situations where the aromatic ring is sufficiently electrophilic in nature to allow the displacement to occur under relatively mild conditions (i.e. minimal heating requirements).

Preparation of Racemic(1R,2S)/(1S,2R)-1-amino-2-vinylcyclopropane carboxylic acid ethyl ester:

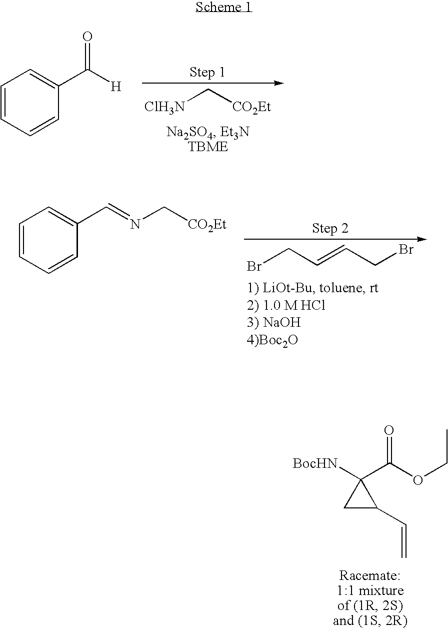

### Step 1:

Glycine ethyl ester hydrochloride (304 g, 2.16 mole) was suspended in tert-butylmethyl ether (1.6 L). Benzaldehyde (231 g, 2.16 mole) and anhydrous sodium sulfate (155 g, 1.09 mole) were added, and the mixture was cooled to 0° C. using an ice-water bath. Triethylamine (455 mL, 3.26 mole) was added dropwise over 30 min and the mixture was stirred for 48 h at rt. The reaction was then quenched by addition of ice-cold water (1 L) and the organic layer was separated. The aqueous phase was extracted with tert-butylmethyl ether (0.5 L) and the organic phases were combined and washed with a mixture of saturated aqueous NaHCO₃ (1 L) and brine (1 L). The organic was dried over MgSO₄ and concentrated in vacuo to afford 392.4 g of the N-benzyl imine product as a thick yellow oil that was used directly in the next step. ¹H NMR (CDCl₃, 300 MHz) δ 1.32 (t, J=7.1 Hz, 3H), 4.24 (q, J=7.1 Hz, 2H), 4.41 (d, J=1.1 Hz, 2H), 7.39-7.47 (m, 3H), 7.78-7.81 (m, 2H), 8.31 (s, 1 H).

### Step 2:

To a suspension of lithium tert-butoxide (84.1 g, 1.05 mol) in dry toluene (1.2 L), was added dropwise a mixture of the N-benzyl imine of glycine ethyl ester (100 g, 0.526 mol) and trans-1,4-dibromo-2-butene (107 g, 0.500 mol) in dry toluene (0.6 L) over 60 min. Upon completion of the addition, the deep red mixture was quenched by addition of water (1 L) and tert-butylmethyl ether (TBME, 1 L). The aqueous phase was separated and extracted a second time with TBME (1 L). The organic phases were combined, 1.0M HCl (1 L) was added and the mixture stirred at room temperature for 2 h. The organic phase was separated and extracted with water (0.8 L). The aqueous phases were then combined, saturated with salt (700 g), and TBME (1 L) was added and the mixture was cooled to 0° C. The stirred mixture was then made basic to pH=14 by the dropwise addition of 10.0M NaOH, the organic layer was separated, and the aqueous phase was extracted with TBME (2×500 mL). The organic extracts were combined, dried over MgSO₄, filtered and concentrated to a volume of 1 L. To this solution of free amine was added Boc₂O or di-tert-butyldicarbonate (131 g, 0.600 mol) and the mixture stirred for 4 d at rt. Additional di-tert-butyldicarbonate (50 g, 0.23 mol) was added to the reaction and the mixture was refluxed for 3 h and was then allowed cool to rt overnight. The reaction mixture was dried over MgSO₄, filtered and concentrated in vacuo to afford 80 g of crude material. This residue was purified by flash chromatography (2.5 kg of SiO₂, eluted with 1% to 2% MeOH/CH₂Cl₂) to afford 57 g (53%) of racemic N-Boc-(1R,2S)/(1S,2R)-1-amino-2-vinylcyclopropane carboxylic acid ethyl ester as a yellow oil which solidified while sitting in the refrigerator: ¹H NMR (CDCl₃, 300 MHz) δ 1.26 (t, J=7.1 Hz, 3H), 1.46 (s, 9H), 1.43-1.49 (m, 1H), 1.76-1.82 (br m, 1H), 2.14 (q, J=8.6 Hz, 1H), 4.18 (q, J=7.2 Hz, 2H), 5.12 (dd J=10.3, 1.7 Hz, 1H), 5.25 (br s, 1H), 5.29 (dd, J=17.6, 1.7 Hz, 1H), 5.77 (ddd, J=17.6, 10.3, 8.9 Hz, 1H); MS m/z 254.16 (M−1).

### Resolution of NV-Boc-(1R,2S)/(1S,2R)-1-amino-2-vinylcyclopropane carboxylic acid ethyl ester

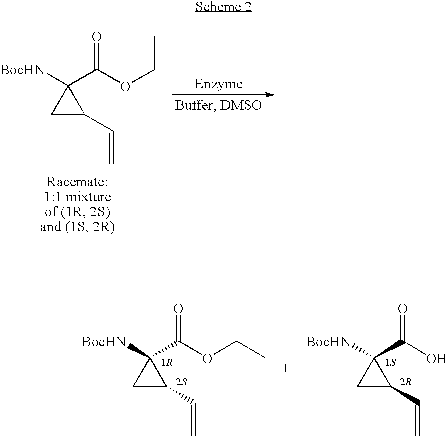

### Resolution A

To an aqueous solution of sodium phosphate buffer (0.1 M, 4.25 liter (“L”), pH 8) housed in a 12 L jacked reactor, maintained at 39° C., and stirred at 300 rpm was added 511 grams of Acalase 2.4 L (about 425 mL) (Novozymes North America Inc.). When the temperature of the mixture reached 39° C., the pH was adjusted to 8.0 by the addition of a 50% NaOH in water. A solution of the racemic N-Boc-(1R,2S)/(1S,2R)-1-amino-2-vinylcyclopropane carboxylic acid ethyl ester (85 g) in 850 mL of DMSO was then added over a period of 40 min. The reaction temperature was then maintained at 40° C. for 24.5 h during which time the pH of the mixture was adjusted to 8.0 at the 1.5 h and 19.5 h time points using 50% NaOH in water. After 24.5 h, the enantio-excess of the ester was determined to be 97.2%, and the reaction was cooled to room temperature (26° C.) and stirred overnight (16 h) after which the enantio-excess of the ester was determined to be 100%. The pH of the reaction mixture was then adjusted to 8.5 with 50% NaOH and the resulting mixture was extracted with MTBE (2×2 L). The combined MTBE extract was then washed with 5% NaHCO₃ (3×100 mL), water (3×100 mL), and evaporated in vacuo to give the enantiomerically pure N-Boc-(1R,2S)/-1-amino-2-vinylcyclopropane carboxylic acid ethyl ester as light yellow solid (42.55 g; purity: 97% @210 nm, containing no acid; 100% enantiomeric excess (“ee”).

The aqueous layer from the extraction process was then acidified to pH=2 with 50% H₂SO₄ and extracted with MTBE (2×2 L). The MTBE extract was washed with water (3×100 mL) and evaporated to give the acid as light yellow solid (42.74 g; purity: 99% @210 nm, containing no ester).

| | ester | acid |
|---|---|---|
| High | (+) ESI, C13H22NO4, | (−) ESI, C11H16NO4, |
| Resolution on | [M + H]⁺, cal. | [M − H]⁻, |
| Mass Spec | 256.1549, found 256.1542 | cal. 226.1079, found |
| | | 26.1089 |

NMR observed chemical shift
Solvent: CDCl₃ (proton δ 7.24 ppm, C-13 δ 77.0 ppm)
Bruker DRX-500C: proton 500.032 MHz, carbon 125.746 MHz

| | Proton (pattern) | C-13 | Proton (pattern) | C-13 |
|---|---|---|---|---|
| Position | ppm | ppm | ppm | ppm |
| 1 | — | 40.9 | — | 40.7 |
| 2 | 2.10 (q, J = 9.0 Hz) | 34.1 | 2.17 (q, J = 9.0 Hz) | 35.0 |
| 3a | 1.76 (br) | 23.2 | 1.79 (br) | 23.4 |
| 3b | 1.46 (br) | | 1.51, (br) | |
| 4 | — | 170.8 | — | 175.8 |
| 5 | 5.74 (ddd, J = 9.0, 10.0, | 133.7 | 5.75 (m) | 133.4 |
| | 17.0 Hz) | | | |
| 6a | 5.25 (d, J = 17.0 Hz) | 117.6 | 5.28 | 118.1 |
| | | | (d, J = 17.0 Hz) | |
| 6b | 5.08 (dd, J = 10.0, 1.5 Hz) | | 5.12 | |
| | | | (d, J = 10.5 Hz) | |
| 7 | — | 155.8 | — | 156.2 |
| 8 | — | 80.0 | — | 80.6 |
| 9 | 1.43 (s) | 28.3 | 1.43 (s) | 28.3 |
| 10 | 4.16 (m) | 61.3 | — | — |
| 11 | 1.23 (t, J = 7.5 Hz) | 14.2 | — | — |

### Resolution B

To 0.5 mL 100 mM Heps•Na buffer (pH=8.5) in a well of a 24 well plate (capacity: 10 mL/well), 0.1 mL of Savinase 16.0 L (protease from Bacillus clausii) (Novozymes North America Inc.) and a solution of the racemic N-Boc-(1R,2S)/(1S,2R)-1-amino-2-vinylcyclopropane carboxylic acid ethyl ester (10 mg) in 0.1 mL of DMSO were added. The plate was sealed and incubated at 250 rpm at 40° C. After 18 h, enantio-excess of the ester was determined to be 44.3% as following: 0.1 mL of the reaction mixture was removed and mixed well with 1 mL ethanol; after centrifugation, 10 microliter (“μl”) of the supernatant was analyzed with the chiral HPLC. To the remaining reaction mixture, 0.1 mL of DMSO was added, and the plate was incubated for additional 3 d at 250 rpm at 40° C., after which 4 mL of ethanol was added to the well. After centrifugation, 10 μl of the supernatant was analyzed with the chiral HPLC and enantio-excess of the ester was determined to be 100%.

Resolution C

To 0.5 mL 100 mM Heps•Na buffer pH=8.5) in a well of a 24 well plate (capacity: 10 mL/well), 0.1 ml of Esperase 8.0 L, (protease from Bacillus halodurans) (Novozymes North America Inc.) and a solution of the racemic N-Boc-(1R,2S)/(1S,2R)-1-amino-2-vinylcyclopropane carboxylic acid ethyl ester (10 mg) in 0.1 mL of DMSO were added. The plate was sealed and incubated at 250 rpm at 40° C. After 18 h, enantio-excess of the ester was determined to be 39.6% as following: 0.1 mL of the reaction mixture was removed and mixed well with 1 mL ethanol; after centrifugation, 10 μl of the supernatant was analyzed with the chiral HPLC. To the remaining reaction mixture, 0.1 mL of DMSO was added, and the plate was incubated for addition 3 d at 250 rpm at 40° C., after which 4 mL of ethanol was added to the well. After centrifugation, 10 μl of the supernatant was analyzed with the chiral HPLC and enantio-excess of the ester was determined to be 100%.
Samples analysis was carried out in the following manner:
1) Sample preparation: About 0.5 mL of the reaction mixture was mixed well with 10 volumes of EtOH. After centrifugation, 10 μl of the supernatant was injected onto HPLC column.

2) Conversion determination:
Column; YMC ODS A, 4.6×50 mm, S-5 μm
Solvent: A=1 mM HCl in water; B=MeCN
Gradient: 30% B for 1 min; 30% to 45% B over 0.5 min; 45% B for 1.5 min; 45% to 30% B over 0.5 min.
Flow rate: 2 mL/min
UV Detection: 210 nm
Retention time: acid, 1.2 min; ester, 2.8 min.
3) Enantio-excess determination for the ester:
Column: CHIRACEL OD-RH, 4.6×150 mm, S-5 μm
Mobile phase: MeCN/50 mM HCl in water (67/33)
Flow rate: 0.75 mL/min.
UV Detection: 210 nm.
Retention time:
(1S, 2R) isomer as acid: 5.2 min;
Racemate: 18.5 min and 20.0 min;
(1R, 2S) isomer as ester: 18.5 min.

### Preparation of Cyclopropylsulfonamide

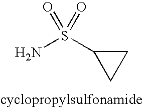

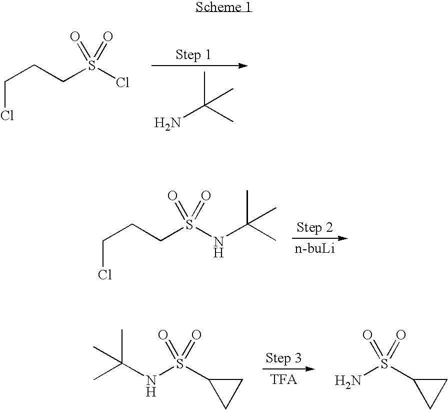

### Step 1:

tert-Butylamine (3.0 mol, 315 mL) was dissolved in THF (2.5 L). The solution was cooled to −20° C. 3-Chloropropanesulfonyl chloride (1.5 mol, 182 mL) was added slowly. The reaction mixture was allowed to warm to rt and stirred for 24 h. The mixture was filtered, and the filtrate was concentrated in vacuo. The residue was dissolved in CH₂Cl₂ (2.0 L). The resulting solution was washed with 1.0M HCl (1.0 L), water (1.0 L), brine (1.0 L), dried over Na₂SO₄, filtered and concentrated in vacuo to give a slightly yellow solid, which was crystallized from hexane to afford the product as a white solid (316.0 g, 99%). ¹H NMR (CDCl) δ 1.38 (s, 9H), 2.30-2.27 (m, 2H), 3.22 (t, J=7.35 Hz, 2H), 3.68 (t, J=6.2 Hz, 2H), 4.35 (1H).

### Step 2:

To a solution of N-tert-butyl-(3-chloro)propylsulfonamide (2.14 g, 10.0 mmol) in THF (100 mL) was added n-BuLi (2.5 M in hexane, 8.0 mL, 20.0 mmol) at −78° C. The reaction mixture was allowed to warm up to room temperature over period of 1 h. The volatiles were removed in vacuo. The residue was partitioned between EtOAc and water (200 mL each). The separated organic phase was washed with brine, dried over Na₂SO₄, filtered and concentrated in vacuo. The residue was recrystallized from hexane to yield the desired product as a white solid (1.0 g, 56%).

¹H NMR (CDCl₃) δ 0.98-1.00 (m, 2H), 1.18-1.19 (m, 2H), 1.39 (s, 9H), 2.48-2.51 (m, 1H), 4.19 (b, 1H).

### Step 3:

A solution of cyclopropanesulfonic acid tert-butylamide (110 g, 0.62 mmol) in TFA (500 mL) was stirred at room temperature for 16 h. The volatiles were removed in vacuo. The residue was recrystallized from EtOAc/hexane (60 mL/240 mL) to yield the desired product as a white solid (68.5 g, 91%). ¹H NMR (DMSO-d₆) δ 0.84-0.88 (m, 2H), 0.95-0.98 (m, 2H), 2.41-2.58 (m, 1H), 6.56 (b, 2H).

### Preparation of P1P1′:

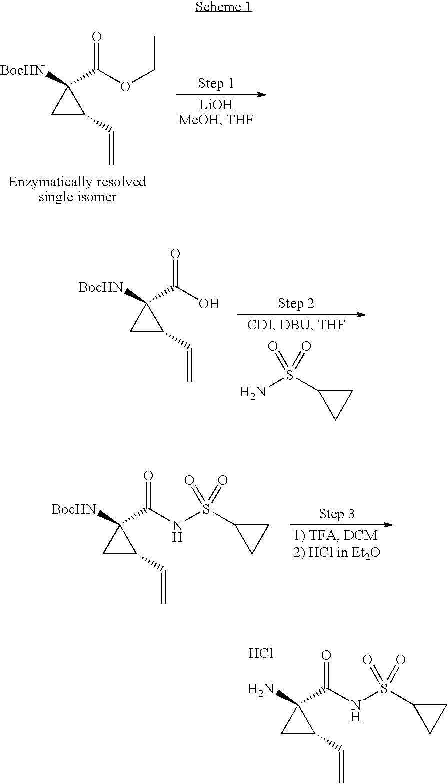

### Step 1:

To a solution of 1(R)-tert-butoxycarbonylamino-2(S)-vinyl-cyclopropanecarboxylic acid ethyl ester (3.28 g, 13.2 mmol) in THF (7 mL) and methanol (7 mL) was added a suspension of LiOH (1.27 g, 53.0 mmol) in water (14 mL). The mixture was stirred overnight at room temperature. To the mixture was added 1.0M NaOH (15 mL), water (20 mL) and EtOAc (20 mL). The mixture was shaken, the phases were separated, and the organic phase was again extracted with 20 mL 0.5M NaOH. The combined aqueous phases were acidified with 1.0M HCl until pH=4 and extracted with EtOAc (3×40 mL). The combined organic extracts were washed with brine and dried (MgSO₄) to yield the title compound as a white solid (2.62 g, 87%). ¹H NMR: (DMSO-d₆) δ1.22-1.26 (m, 1H), 1.37 (s, 9H), 1.50-1.52 (m, 1H), 2.05 (q, J=9 Hz, 1H), 5.04 (d, J=10 Hz, 1H), 5.22 (d, J=17 Hz, 1H), 5.64-5.71 (m, 1H), 7.18, 7.53 (s, NH (rotamers), 12.4 (br s, 1H)); LC-MS MS m/z 228 (M⁺+H).

### Step 2:

A solution of the product of Step 1 (2.62 g, 11.5 mmol) and CDI (2.43 g, 15.0 mmol) in THF (40 mL) was heated at reflux for 50 min under nitrogen. The solution was cooled to room temperature and transferred by cannula to a solution of cyclopropylsulfonamide (1.82 g, 15.0 mmol) in THF (10 mL). To the resulting solution was added DBU (2.40 mL, 16.1 mmol) and stirring was continued for 20 h. The mixture was quenched with 1.0M HCl to pH=1, and THF was evaporated in vacuo. The suspension was extracted with EtOAc (2×50 mL) and the organic extracts were combined and dried (Na₂SO₄). Purification by recrystallization from hexanes-EtOAc (1:1) afforded the title compound (2.4 g) as a white solid. The mother liquor was purified by a Biotage 40S column (eluted 9% acetone in DCM) to give a second batch of the title compound (1.1 g). Both batches were combined (total yield 92%). ¹H NMR: (DMSO-d₆) δ 0.96-1.10 (m, 4H), 1.22 (dd, J=5.5, 9.5 Hz, 1H), 1.39 (s, 9H), 1.70 (t, J=5.5 Hz, 1H), 2.19-2.24 (m, 1H), 2.90 (m, 1H), 5.08 (d, J=10 Hz, 1H), 5.23 (d, J=17 Hz, 1H), 5.45 (m, 1H), 6.85, 7.22 (s, NH (rotamers)); LC-MS, MS m/z 331 (M⁺+H).

### Step 3:

A solution of the product of Step 2 (3.5 g, 10.6 mmol) in DCM (35 mL) and TFA (32 mL) was stirred at room temperature for 1.5 h. The volatiles were removed in vacuo and the residue suspended in 1.0M HCl in diethyl ether (20 mL) and concentrated in vacuo. This procedure was repeated once. The resulting mixture was triturated with pentane and filtered to give the title compound as a hygroscopic, off-white solid (2.60 g, 92%). ¹H NMR (DMSO-d₆) δ 1.01-1.15 (m, 4H), 1.69-1.73 (m, 1H), 1.99-2.02 (m, 1H), 2.38 (q, J=9 Hz, 1H), 2.92-2.97 (m, 1H), 5.20 (d, J=11 Hz, 1H), 5.33 (d, J=17 Hz, 1H), 5.52-5.59 (m, 1H), 9.17 (br s, 3H); LC-MS, MS m/z 231 (M⁺+H).

### EXAMPLE 1

### Preparation of Compounds 1A and 1B

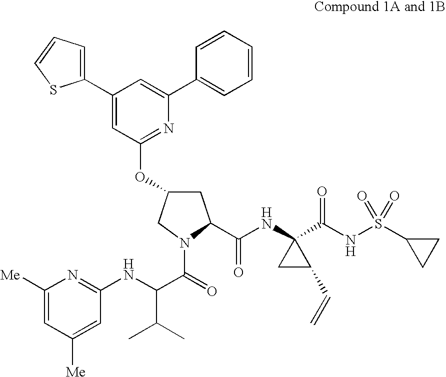

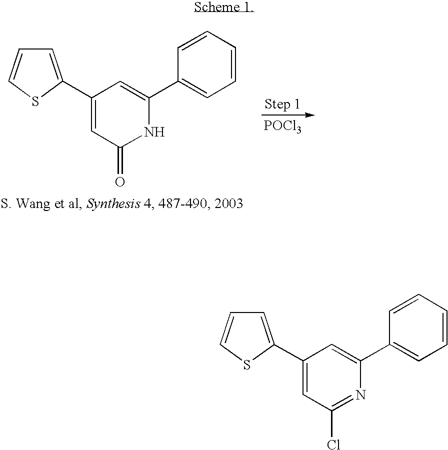

### Step 1.

A solution of 6-phenyl-4-(thiophen-2-yl)pyridin-2(1H)-one (1.07 mg, 4.23 mmol) (prepared according to S. Wang et al., Synthesis 4, 487-490, 2003) in phosphorus oxychloride (15 mL) was heat to reflux for three days. The excess phosphorus oxychloride was removed in vacuo and the residue was triturated with ice-water. The triturant was made basic with aqueous NaOH and the product was extracted into DCM. The organic layer was washed with brine, dried, filtered through celite and evaporated. Crude product was purified by flash column chromatography to give a white solid product (624 mg, 54% yield). ¹H NMR (CDCl₃) δ ppm 7.16 (dd, J=5.13, 3.7 Hz, 1H), 7.44-7.52 (m, 5H), 7.55 (dd, J=3.7, 1.1 Hz, 1H), 7.79 (d, J=1.5 Hz, 1H), 8.02 (dd, J=8.1, 1.5 Hz, 2H); LC-MS, MS m/z 272 (M⁺+H).

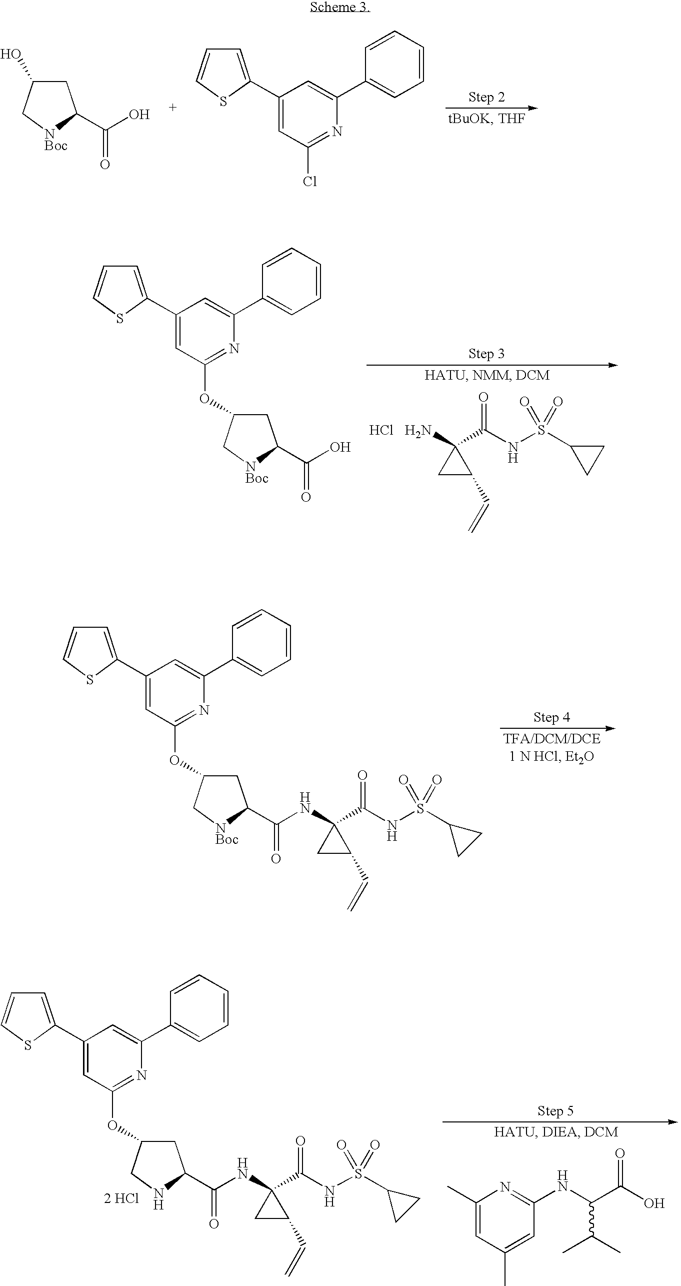

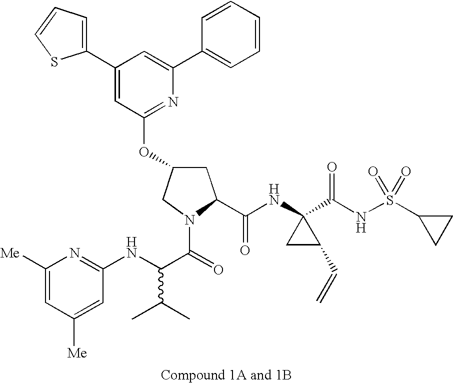

### Step 2.

To a solution of Boc-Hyp-OH (254 mg, 1.1 mmol) in DMSO (5 mL) was added potassium tert-butoxide (295 mg, 2.5 mmol). After stirring at rt for 1 h, the chloropyridine product from step 1, Example 1 was added and the resulting mixture was stirred at rt overnight. The reaction mixture was partitioned between EtOAc and aqueous citric acid. The organic phase was washed with H₂O and brine, and was then dried over MgSO₄ and evaporated in vacuo. LC/MS of crude mixture showed a 2.5:1 mixture of product:chloropyridine starting material. The crude mixture was purified by a flash column chromatography (SiO₂, 90:10 DCM:MeOH) to give a solid product (270 mg, 58% yield). ¹H NMR (CD₃OD) δ 1.45 (s, 9H), 2.37-2.42 (m, 1H), 2.63 (q, J=13.9 Hz, 1H), 3.79 (d, J=11.9 Hz, 1H), 3.88 (d, J=12.2 Hz, 1H), 4.41-4.46 (m, 1H), 5.70 (br s, 1H), 6.92 (br s, 1H), 7.15 (d, J=3.4 Hz, 1H), 7.40 (t, J=6.1 Hz, 1H), 7.45 (q, J=6.7 Hz, 2H), 7.51 (d, J=4.0 Hz, 1H), 7.65 (br s, 2H), 8.05 (d, J=7.0 Hz, 2H); LC-MS, MS m/z 467 (M⁺+H).

### Step 3.

The product from step 2, Example 1, (260 mg, 0.56 mmol) was combined with N-methylmorpholine (284 mg, 2.79 mmol), cyclopropanesulfonic acid (1-(R)-amino-2-(S)-vinyl-cyclopropanecarbonyl)-amide HCl salt (202 mg, 0.61 mmol) and HATU (276 mg, 0.73 mmol) in DCM (5 mL). After stirring at rt for 2 h, the reaction mixture was poured into aqueous citric acid and the product was extracted with EtOAc. The organic layer was washed with aqueous bicarbonate, and brine, and was then dried over MgSO₄ and evaporated in vacuo. The crude mixture was purified by flash column chromatography (SiO₂, 1.5% MeOH in DCM) to give a white solid product (250 mg, 66% yield). NMR (CD₃OD) δ 1.07 (q, J=7.1 Hz, 2H), 1.18 (dd J=9.5, 4.3 Hz, 1H), 1.23-1.29 (m, 1H), 1.43 (q, J=6.1 Hz, 1H), 1.47 (s, 9H), 1.88 (q, J=5.5 Hz, 1H), 2.25 (q, J=8.5 Hz, 1H), 2.30 (dd, J=9.5, 4.6 Hz, 1H), 2.51 (dd, J=13.5 Hz, 1H), 2.93-2.97 (m, 1H), 3.77 (d, J=11.9 Hz, 1H), 3.89 (dd, J=11.6, 4.1 Hz, 1H), 4,32 (t, J=8.3 Hz, 1H), 5.12 (d, J=10.4 Hz, 1H), 5.31 (d, J=17.1 Hz, 1H), 5.76 (br s, 1H), 6.93 (s, 1H), 7.16 (t, J=4.3 Hz, 1H), 7.41 (t, J=6.9 Hz, 1H), 7.46 (t, J=7.5 Hz, 2H), 7.54 (d, J=4.9 Hz, 1H), 7.68 (br s, 2H), 8.06 (d, J=7.6 Hz, 2H); LC-MS, MS m/z 678 (M⁺+H).

### Step 4.

To a solution of the product of step 3, Example 1, (0.707 g, 1.04 mmol) in 1:1 DCM:DCE (20 mL) was added TFA (10 mL). After stirring at rt for 0.5 h, the reaction was concentrated in vacuo. The resulting residue was re-dissolved in DCE (20 mL) and re-concentrated. The resulting brown vicous oil was then dissolved in DCM (3 mL) and was added dropwise to a rapidly stirred solution of 1N HCl in Et₂O (100 mL). The resulting precipitate, an off-white solid (0.666 g, 98% yield) was obtained by vacuum filtration and was washed with Et₂O. LC-MS, MS m/z 579 (M⁺+H).

### Step 5.

To a mixture of product the product of Step 4, Example 1, (240.0 mg, 0.368 mmol), DIEA (0.277 g, 2.14 mmol) and (±)-2-(4,6-dimethylpyridin-2-ylamino)-3-methylbutanoic acid (0.135 g, 0.610 mmol, purchased from Specs, catalog #AP-836/41220382) in DCM (4 mL) was added HATU (210.1 mg, 0.552 mmol). The reaction was stirred at rt for 8 h. Additional HATU (070.0 mg, 0.184 mmol), (±)-2-(4,6-dimethylpyridin-2-ylamino)-3-methylbutanoic acid (0.141.0 mg, 0.0.184 mmol) were added and the resulting mixture was stirred for an additional 8 h in an attempt to push the reaction further towards completion. The mixture was concentrated in vacuo, dissolved in EtOAc (50 mL), and washed with 1.0M aqueous HCl (2×5 mL). The combined HCl washes were back-extracted with EtOAc (50 mL). The organics were combined and washed with 10% aqueous NaHCO₃ (50 mL) and with brine, and were then dried over MgSO₄, filtered and concentrated. Purification by reverse phase preparative HPLC (Sunfire prep-HPLC column, solvent A=H₂O with 0.1% TFA, solvent B=MeOH with 0.1% TFA, 30 minutes gradient: started with 15% A to 100% B) gave two products with identical m/z by LCMS. HPLC fractions for each product were combined and concentrated, treated with 1N HCl and MeOH then re-concentrated and dried under vacuo to give a mono HCl salt product. The first isomer to elute by reverse phase preparative HPLC was labeled Compound 1A (91.0 mg, 28.9%) and the second isomer to elute was labeled Compound 1B (16.9 mg, 5.4%).

Compound 1A: ¹H NMR (500 MHz, MeOD) δ ppm 1.01 (d, J=6.7 Hz, 3H), 1.09 (d, J=6.7 Hz, 3H), 1.11-1.17 (m, 2H), 1.20-1.32 (m, 2H), 1.44-1.49 (m, 1H), 1.94 (dd, J=8.2, 5.5 Hz, 1H), 2.18-2.24 (m, 3H), 2.25-2.35 (m, 2H), 2.34-2.38 (m, 3 H), 2.39-2.49 (m, 2H), 2.63 (dd, J=13.4, 7.0 Hz, 1H), 2.94-3.03 (m, 1H), 4.15-4.28 (m, 2H), 4.52 (d, J=7.6 Hz, 1H), 4.58-4.65 (m, 1H), 5.17 (d, J=10.4 Hz, 1H), 5.36 (d, J=17.1 Hz, 1H), 5.73-5.86 (m, 1H), 6.00 (s, 1H), 6.59 (s, 1H), 6.75 (s, 1H), 6.90-6.93 (m, 1H), 7.17-7.23 (m, 1H), 7.44-7.55 (m, 3H), 7.56-7.62 (m, 1H), 7.70-7.80 (m, 2H), 8.15 (d, J=7.3 Hz, 2H)LC-MS, MS m/z 783 (M⁺+H).

Compound 1B: ¹H NMR (500 MHz, MeOD) δ ppm 0.96 (d, J=6.4 Hz, 6H), 1.00-1.14 (m, 4H), 1.30-1.38 (m, 1H), 1.39-1.45 (m, 1H), 1.93 (dd, J=8.1, 5.3 Hz, 1H), 2.03-2.15 (m, 1H), 2.32 (q, J=8.7 Hz, 1H), 2.40-2.42 (m, 3H), 2.43-2.48 (m, 3H), 2.58-2.67 (m, 1H), 2.82-2.92 (m, 2H), 4.19-4.28 (m, 1H), 4.62 (dd, J=16.6, 7.2 Hz, 2H), 5.17 (d, J=11.9 Hz, 1H), 5.35 (d, J=17.1 Hz, 1H), 5.73-5.83 (m, 1H), 6.01 (s, 1H), 6.69 (s, 1H), 6.83 (s, 1H), 6.96 (s, 1H), 7.18-7.25 (m, 1H), 7.43-7.55 (m, 3H), 7.57-7.63 (m, 1H), 7.72-7.77 (m, 1H), 7.80 (s, 1H), 8.15 (d, J=7.3 Hz, 2H), 9.53 (s, 1H); LC-MS, MS m/z 783 (M⁺+H).

### EXAMPLE 2

### Preparation of Compound 2

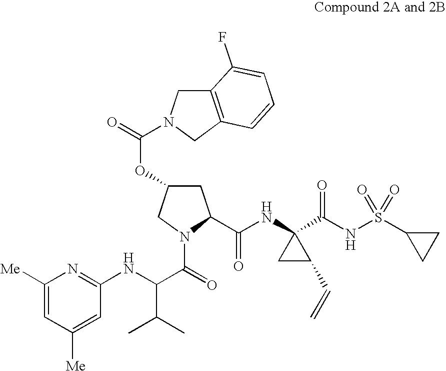

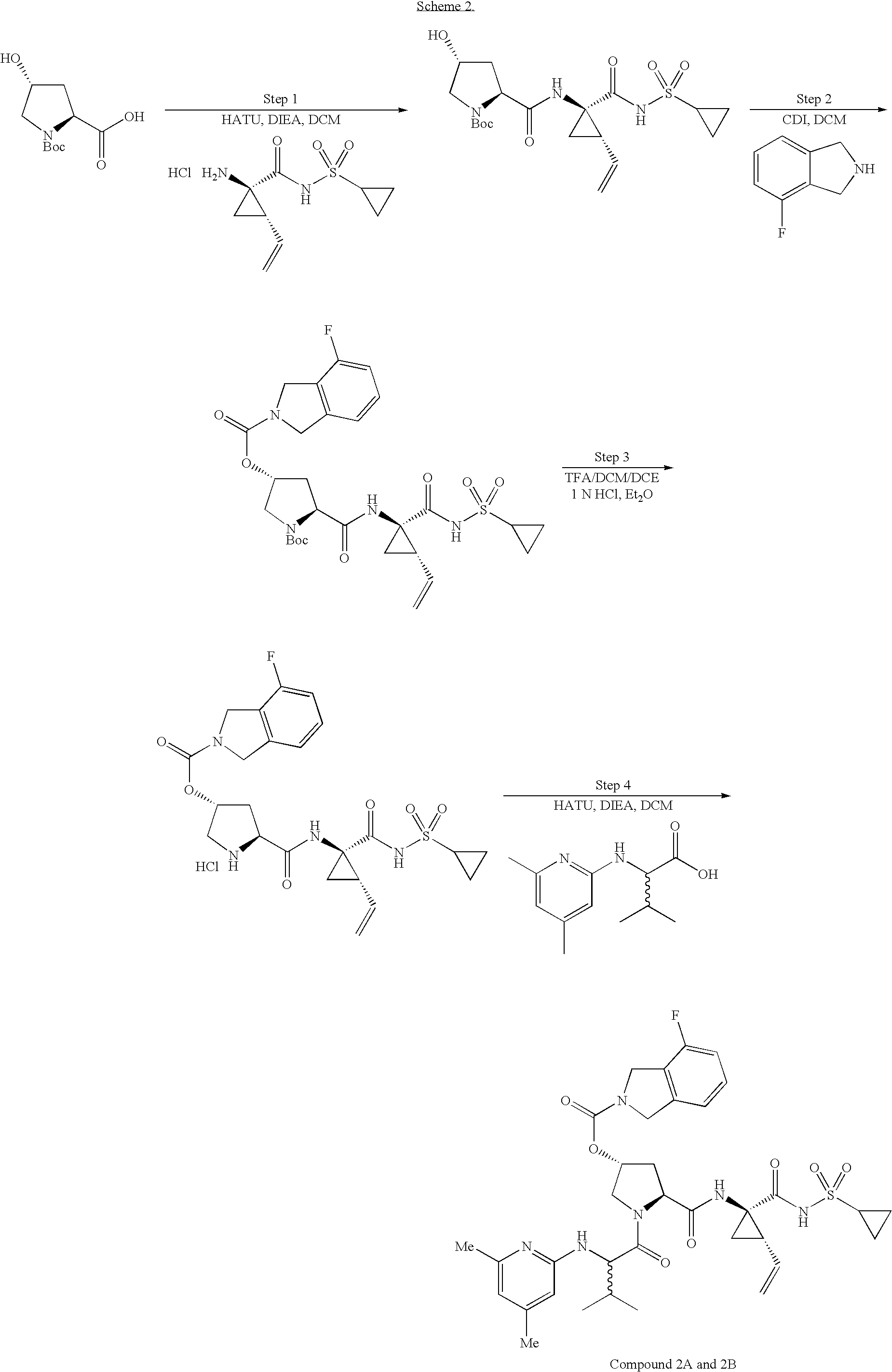

### Step 1.

The product of step 1, Example 2, was prepared by the same procedure as the product of step 3, Example 1, starting with Boc-Hyp-OH instead of the product of step 2, Example 1. ¹H NMR (500 MHz, MeOD) δ ppm 1.09 (d, J=7.63 Hz, 2H) 1.16-1.22 (m, 1H) 1.25-1.32 (m, 1H) 1.42 (dd, J=9.46, 5.49 Hz, 1H) 1.47 (s, 1.7H) 1.50 (s, 7.3H) 1.88 (dd, J=8.09, 5.34 Hz, 1H) 1.94-2.03 (m, 1H) 2.13 (dd, J=12.97, 6.87 Hz, 1H) 2.26 (q, J=8.85 Hz, 1H) 2.97 (ddd, J=12.51, 8.09, 4.73 Hz, 1H) 3.47 (d, J=11.60 Hz, 1H) 3.56-3.62 (m, 1H) 4.25 (dd, J=9.61, 6.87 Hz, 1H) 4.42 (s, 1H) 5.15 (d, J=10.38 Hz, 1H) 5.34 (d, J=17.09 Hz, 1H) 5.74-5.85 (m, 1H); LCMS, MS m/z=442 (M−H)⁻.

### Step 2.

To a solution of the product from step 1, Example 2, (1.0 g, 2.25 mmol) in DCM (20 mL) was added 1,1′-carbonyldiimidazole (439 mg, 2.71 mmol). After stirring at rt for 3 h, 4-fluoroisoindoline (prepared according to procedure found in: L. M. Blatt et al. PCT Int. Appl. (2005), 244 pp, WO 2005037214) (617 mg, 4.50 mmol) was added and the resulting mixture was stirred at rt overnight. The reaction mixture was diluted with EtOAc (100 mL) and washed with 2×10 mL 1N aqueous HCl. The aqueous layer was extracted with 2×50 mL EtOAc. The combined organic layer was washed with brine, dried over MgSO₄, and concentrated to a dark brown viscous oil. The crude mixture was purified by flash column chromatography (SiO₂, 97:3 and 95:5 DCM:MeOH) to give a grey foamy solid (1.3 g, 95% yield). ¹H NMR (500 MHz, CHLOROFORM-D) δ ppm 1.29-1.37 (m, 2H) 1.38-1.45 (m, 2H) 1.47 (s, 9H) 1.95-2.00 (m, 1H) 2.07-2.14 (m, 1H) 2.28-2.35 (m, 1H) 2.37-2.46 (m, 1H) 2.90-2.97 (m, 1H) 3.65 (d, J=12.80 Hz, 1H) 3.72 (d, J=12.50 Hz, 1H) 4.26 (t, J=7.02 Hz, 1H) 4.68 (d, J=9.46 Hz, 2H) 4.77 (d, J=9.16 Hz, 2H) 5.15 (d, J=10.38 Hz, 1H) 5.29 (d, J=17.10 Hz, 1H) 5.33 (s, 1H) 5.73-5.84 (m, 1H) 6.97 (t, J=8.70 Hz, 1H) 7.01 (d, J=7.63 Hz, 1H) 7.28 (dd, J=8.09, 2.90 Hz, 1H) 10.00 (s, 1H); LC-MS , MS m/z 629 (M⁺+Na).

### Step 3.

The product of step 3, Example 2, was prepared in 94% yield from the product of step 2, Example 2, by the same procedure as described for the preparation of the product of step 4, Example 1. ¹H NMR (500 MHz, MeOD) δ ppm 1.05-1.11 (m, 1H) 1.11-1.17 (m, 1H) 1.18-1.23 (m, 1H) 1.27-1.34 (m, 1H) 1.40 (dd, J=9.61, 5.65 Hz, 1H) 1.98 (dd, J=7.93, 5.80 Hz, 1H) 2.27-2.33 (m, 1H) 2.36 (q, J=8.80 Hz, 1H) 2.75 (dd, J=14.34, 7.32 Hz, 1H) 2.96-3.03 (m, 1H) 3.65-3.75 (m, 2H) 4.61-4.67 (m, 1H) 4.78 (s, 2H) 5.19 (d, J=10.38 Hz, 1H) 5.36 (d, J=17.09 Hz, 1H) 5.48 (s, 1H) 5.64-5.73 (m, 1H) 7.06 (t, J=8.70 Hz, 1H) 7.17 (dd, J=16.17, 7.63 Hz, 1H) 7.37 (q, J=7.63 Hz, 1H); LC-MS , MS m/z 507 (M⁺+H).

### Step 4.

The product of step 4, Example 2, was prepared in 24.9% yield for Compound 2A and 8.4% yield for Compound 2B from the product of step 3, Example 2, by the same procedure as described for the preparation of the product of step 5, Example 1.

Compound 2A: ¹H NMR (500 MHz, MeOD) δ ppm 1.03 (d, J=4.9 Hz, 3H), 1.11 (d, J=5.5 Hz, 3H), 1.13-1.20 (m, 2H), 1.24-1.30 (m, 2H), 1.45 (dd, J=9.5, 5.2 Hz, 1H), 1.93 (dd, J=8.1, 5.3 Hz, 1H), 2.23-2.32 (m, 2H), 2.35 (s, 3H), 2.48 (s, 3H), 2.49-2.55 (m, 1H), 2.94-3.03 (m, 1H), 4.03-4.10 (m, 1H), 4.20 (d, J=12.2 Hz, 1H), 4.54 (t, J=7.6 Hz, 1H), 4.62 (d, J=6.4 Hz, 1H), 4.67 (s, 1H), 4.71-4.78 (m, 4H), 5.17 (d, J=10.1 Hz, 1H), 5.35 (d, J=17.1 Hz, 1H), 5.50 (d, J=3.7 Hz, 1H), 5.75-5.86 (m, 1H), 6.67 (s, 1H), 6.86 (s, 1H), 7.18 (d, J=7.6 Hz, 2H), 7.32-7.41 (m, 1H); LC-MS, MS m/z 711 (M⁺+H).

Compound 2B: ¹H NMR (500 MHz, MeOD) δ ppm 1.03-1.13 (m, 8H), 1.26-1.32 (m, 1H), 1.38-1.42 (m, 1H), 1.91 (dd, J=8.1, 5.3 Hz, 1H), 2.20-2.36 (m, 4H), 2.44 (s, 3H), 2.49 (s, 3H), 2.83-2.90 (m, 2H), 2.91-2.99 (m, 1H), 3.14-3.25 (m, 1H), 4.11-4.18 (m, 2H), 4.50-4.56 (m, 1H), 4.65-4.69 (m, 1H), 4.71 (s, 1H), 4,77 (d, J=5.8 Hz, 4H), 5.16 (d, J=11.6 Hz, 1H), 5.35 (d, J=17.1 Hz, 1H, 1H), 5.50 (s, 1H), 5.70-5.81 (m, 1H), 6.72 (s, 1H), 6.85 (s, 1H), 7.05 (d, J=9.2 Hz, 1H), 7.14 (d, J=7.3 Hz, 1H), 7.19 (d, J=7.9 Hz, 1H), 7.33-7.41 (m, 1H); LC-MS, MS m/z 711 (M⁺+H).

### EXAMPLE 3

### Preparation of Compound 3

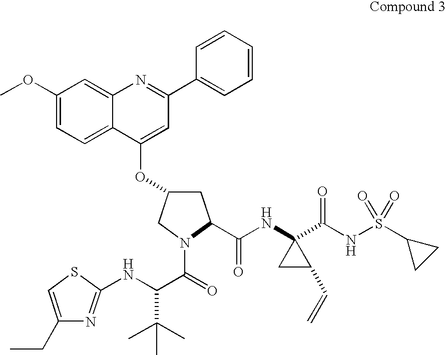

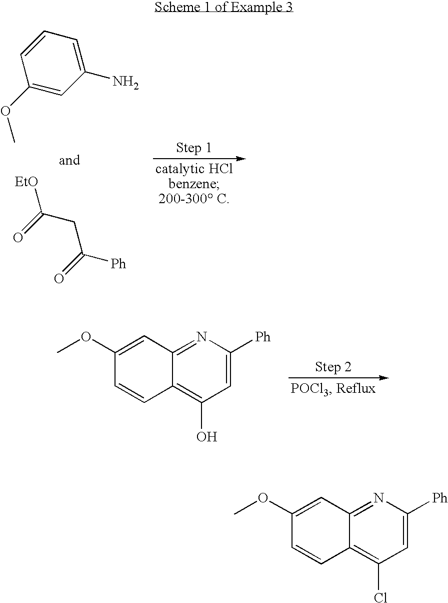

### Step 1:

To a solution of m-anisidine (300 g, 2.44 mol) and ethyl benzoylacetate (234.2 g, 1.22 mol) in toluene (2.0 L) was added HCl (4.0N in dioxane, 12.2 mL, 48.8 mmol). The resulting solution was refluxed for 6.5 hours using a Dean-Stark apparatus (about 56 mL of aqueous solution was collected). The mixture was cooled to room temperature, partitioned multiple times with aqueous HCl (10%, 3×500 mL), aqueous NaOH (1.0N, 2×200 mL), water (3×200 mL), and the organic layer dried (MgSO₄), filtered, and concentrated in vacuo to supply an oily residue (329.5 g). The crude product was heated in an oil bath (280° C.) for 80 minutes using a Dean-Stark apparatus (about 85 mL liquid was collected). The reaction mixture was cooled down to room temperature, the solid residue triturated with CH₂Cl₂ (400 mL), the resulting suspension filtered, and the filter cake washed with more CH₂Cl₂ (2×150 mL). The resulting solid was dried in vacuo (50° C.; 1 torr; 1 day) affording analytically pure product as a light brown solid (60.7 g, 20% overall). ¹H NMR (DMSO-d₆) δ 3.86 (s, 3H), 6.26 (s, 1H), 6.94 (dd, J=9.0, 2.4 Hz, 1H), 7.21 (d, J=2.4 Hz, 1H), 7.55-7.62 (m, 3H), 7.80-7.84 (m, 2H), 8.00 (d, J=9.0 Hz, 1H), 11.54 (s, 1H); ¹³C NMR (DMSO-d₆) δ 55.38, 99.69, 107.07, 113.18, 119.22, 126.52, 127.17, 128.97, 130.34, 134.17, 142.27, 149.53, 161.92, 176.48. LC-MS (MS m/z 252 (M⁺+1).

### Step 2:

The product of Step 1 (21.7 g, 86.4 mmol) was suspended in POCl₃ (240 mL). The suspension was refluxed for 2 hours. After removal of the POCl₃ in vacuo, the residue was partitioned between ethyl acetate (1 L), and cold aqueous NaOH (generated from 1.0N 200 mL NaOH and 20 mL 10.0N NaOH) and stirred for 15 minutes. The organic layer was washed with water (2×200 mL), brine (200 mL), dried (MgSO₄), and concentrated in vacuo to supply the desired product (21.0 g, 90%) as a light brown solid. ¹H NMR (DMSO-d₆) δ 3.97 (s, 3H), 7.36 (dd, J=9.2, 2.6 Hz, 1H), 7.49-7.59 (m, 4H), 8.08 (d, J=9.2 Hz, 1H), 8.19 (s, 1H), 8.26-8.30 (m, 2H); ¹³C NMR (DMSO-d₆) δ 55.72, 108.00, 116.51, 119.52, 120.48, 124.74, 127.26, 128.81, 130.00, 137.58, 141.98, 150.20, 156.65, 161.30. LC-MS (MS m/z 270 (M⁺+1).

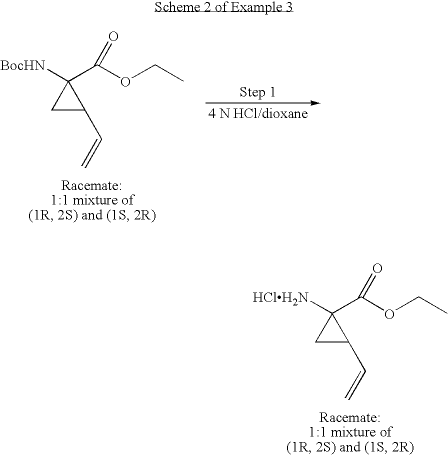

### Step 1:

The racemix mixture of (1R, 2S) and (1S, 2R) of 1-(tert-butoxycarbonylamino)-2-vinylcyclopropanecarboxylate (9.39 g, 36.8 mmol) was dissolved in 4N HCl/dioxane (90 mL, 360 mmol) and was stirred for 2 hours at room temperature. The reaction mixture was concentrated to supply the desired product in quantitative yield (7 g, 100%). ¹H NMR (CD₃OD) δ 1.32 (t, J=7.1, 3H), 1.72 (dd, J=10.2, 6.6 Hz, 1H), 1.81 (dd, J=8.3, 6.6 Hz, 1H), 2.38 (q, J=8.3 Hz, 1H), 4.26-4.34 (m, 2H), 5.24 (dd, 10.3, 1.3 Hz, 1H) 5.40 (d, J=17.2, 1H), 5.69-5.81 (m, 1H).

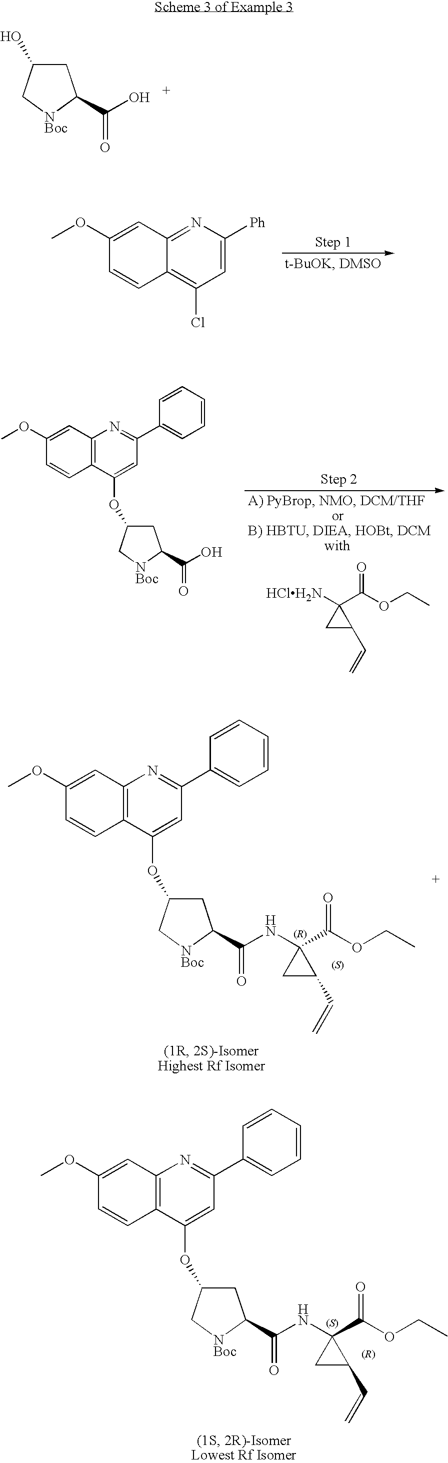

### Step 1:

To a suspension of Boc-4R-hydroxyproline (16.44 g, 71.1 mmol) in DMSO (250 mL) was added t-BuOK (19.93 g, 177.6 mmol) at 0° C. The generated mixture was stirred for 1.5 hours and then the product of Step 2, Scheme 1 (21.02 g, 77.9 mmol) was added in three portions over 1 hour. The reaction was stirred for one day, poured into cold water (1.5 L) and washed with diethyl ether (4×200 mL). The aqueous solution was acidified to pH 4.6, filtered to obtain a white solid, and dried in vacuo to supply the product (32.5 g, 98%). ¹H NMR (DMSO-d₆) δ 1.32, 1.35 (two s (rotamers) 9H), 2.30-2.42 (m, 1H), 2.62-2.73 (m, 1H), 3.76 (m, 2H), 3.91 (s, 3H), 4.33-4.40 (m, 1H), 5.55 (m, 1H), 7.15 (dd, J=9.2, 2.6 Hz, 1H), 7.37 (d, J=2.6 Hz, 1H), 7.42-7.56 (m, 4H), 7.94-7.99 (m, 1H), 8.25, 8.28 (2s, 2H), 12.53 (brs, 1H); LC-MS, MS m/z 465 (M⁺+1).

### Step 2A:

To a solution of the product of Step 1 (11.0 g, 23.7 mmol), the product of Step 1, Scheme 2 (5.40 g, 28.2 mmol), and NMM (20.8 mL; 18.9 mmol) in 500 mL of 50% CH₂Cl₂/THF was added the coupling reagent bromotrispyrrolidinophosphonium hexafluorophosphate (Pybrop) (16.0 g, 34.3 mmol) in three portions in 10 minutes at 0° C. The solution was stirred at room temperature for one day and then was washed with pH 4.0 buffer (4×50 mL). The organic layer was washed with saturated aqueous NaHCO₃ (100 mL), the aqueous wash extracted with ethyl acetate (150 mL), and the organic layer backwashed with pH 4.0 buffer (50 mL) and saturated aqueous NaHCO₃ (50 mL). The organic solution was dried (MgSO₄), filtered, concentrated, and purified by flash column chromatography (SiO₂, eluted with 50% ethyl acetate/hexanes) to provide over 7.5 g of a 1:1 mixture of (1R, 2S) and (1S, 2R) P1 isomers of the desired product (50% overall) or, alternatively, eluted slow with 15% to 60% ethyl acetate in hexanes gradient to supply 3.54 g (25%) of the high Rf eluted (1R, 2S) P1 isomer, and 3.54 g (25%) of the low Rf eluted (1S, 2R) P1 isomer.

Data for (1R, 2S) P1 isomer: ¹H NMR (CDCl₃) δ 1.21 (t, J=7 Hz, 3H), 1.43 (s, 9H), 1.47-1.57 (m, 1H), 1.88 (m, 1H), 2.05-2.19 (m, 1H), 2.39 (m, 1H), 2.88 (m, 1H), 3.71-3.98 (m, 2H), 3.93 (s, 3H), 4.04-4.24 (m, 2H), 4.55 (m, 1H), 5.13 (d, J=10 Hz, 1), 5.22-5.40 (m, 1H), 5.29 (d, J=17 Hz, 1H), 5.69-5.81 (m, 1H), 7.02 (brs, 1H), 7.09 (dd, J=9, 2 Hz, 1H), 7.41-7.52 (m, 4H), 7.95 (d, J=9 Hz, 1H), 8.03, 8.05 (2s, 2H); ¹³C NMR (CDCl₃) δ: 14.22; 22.83, 28.25, 33.14, 33.58, 39.92, 51.84, 55.47, 58.32, 61.30, 75.86, 81.27, 98.14, 107.42, 115.00, 117.84, 118.27, 122.63, 123.03, 127.50, 128.72, 129.26, 133.39, 140.06, 151.23, 159.16, 160.34, 161.35, 169.78, 171.68. LC-MS (MS m/z 602 (M⁺+1).

Data for the (1S, 2R) P1 isomer: ¹H NMR δ 1.25 (t, J=7 Hz, 3H), 1.44 (s, 9H), 1.46-1.52 (m, 1H), 1.84 (m, 1H), 2.12-2.21 (m, 1H), 2.39 (m, 1H), 2.94 (m, 1H), 3.82 (m, 2H), 3.97 (s, 3H), 4.05-4.17 (m, 2H), 4.58 (m, 1H), 5.15 (d, J=10.8 Hz, 1H), 5.33 (d, J=17 Hz, 1H), 5.30-5.43 (m, 1H), 5.72-5.85 (m, 1H), 7.05 (s, 1H), 7.13 (dd, J=9, 2 Hz, 1H), 7.46-7.60 (m, 4H), 7.98 (d, J=9, 1H), 8.06-8.10 (m, 2H). LC-MS MS m/z 602 (M⁺+1).

### Step 2:B:

The product of Step 1, Scheme 2 (7.5 g, 39.1 mmol) was combined with diisopropylethylamine (32.5 mL, 186 mmol) in dichloromethane (150 mL). To the resulting mixture was added HOBT hydrate (6.85 g, 44.7 mmol) and the product from Step 1 (17.3 g, 37.3 mmol), followed by HBTU (16.96 g, 44.7 mmol). A slight exotherm occurred immediately, and the mixture was stirred at room temperature overnight. The mixture was then concentrated in vacuo and redissolved in ethyl acetate (600 mL). The solution was washed with water (2×200 mL), then with 10% aqueous sodium bicarbonate (2×200 mL), then with water (150 mL) and finally with brine (150 mL). The organic was dried over anhydrous magnesium sulfate and filtered, and the filtrate was concentrated in vacuo to a beige glassy solid. Purification was performed in multiple batches (7 g each) by flash chromatography (SiO₂, eluted with 66% hexanes/ethyl acetate) to provide the (1R, 2S) P1 isomer as the initial eluted isomer (9.86 g total, 44.0% yield), followed by elution of the (1S, 2R) P1 isomer as the second eluted isomer (10.43 g total, 46.5% yield). A total of 1.97 g of mixed fractions were recovered to give an overall conversion of 99.3% to the two diastereomers.

Data for (1R, 2S) P1 isomer: ¹H NMR (methanol-d₄) δ 1.23 (t, J=7.2 Hz, 3H), 1.4 (s, 4H), 1.45 (s, 6H), 1.73 (dd, J=7.9, 1.5 Hz, 0.4H), 1.79 (dd, J=7.8, 2.4 Hz, 0.6H), 2.21 (q, J=8.2 Hz, 1H), 2.44-2.49 (m, 1H), 2.66-2.72 (m, 0.4H), 2.73-2.78 (m, 0.6H), 3.93-3.95 (m, 2H), 3.96 (s, 3H), 4.10-4.17 (m, 2H), 4.44 (q, J=7.8 Hz, 1H), 5.13 (d, J=10.7 Hz, 1H), 5.31 (d, J=17.7 Hz, 0.4H), 5.32 (d, J=17.4 Hz, 0.6H), 5.49 (bs, 1H), 5.66-5.82 (m, 1H), 7.16 (dd, J=9.2, 2.5 Hz, 1H), 7.26 (s, 1H), 7.42 (d, J=2.4 Hz, 1H), 7.48-7.55 (m, 3H), 8.02-8.05 (m, 3H); LC-MS (MS m/z 602 (M⁺+1).

Data for (1S, 2R) P1 isomer: ¹H NMR (methanol-d₄) δ 1.23 (t, J=7.2 Hz, 3H), 1.40 (s, 3.5H), 1.43 (s, 6.5H), 1.8 (dd, J=7.2, 5.3 Hz, 0.4H), 1.87 (dd, J=7.8, 5.7 Hz, 0.6H), 2.16 (q, J=8.9 Hz, 0.6H), 2.23 (q, J=8.85 Hz, 0.4H), 2.42-2.50 (m, 1H), 2.67-2.82 (m, 1H), 3.87-3.95 (m, 2H), 3.96 (s, 3H), 4.07-4.19 (m, 3H), 4.41-4.47 (m, 1H), 5.09-5.13 (m, 1H), 5.30 (dd, J=17.09, 0.92 Hz, 1H), 5.48 (s, 1H), 5.70-5.77 (m, 1H), 7.15 (dd, J=9.16, 2.44 Hz, 1H), 7.25 (s, 1H), 7.41 (d, J=2.14 Hz, 1H), 7.48-7.55 (m, 3H), 8.02-8.05 (m, 3H); LC-MS (MS m/z 602 (M⁺+1).

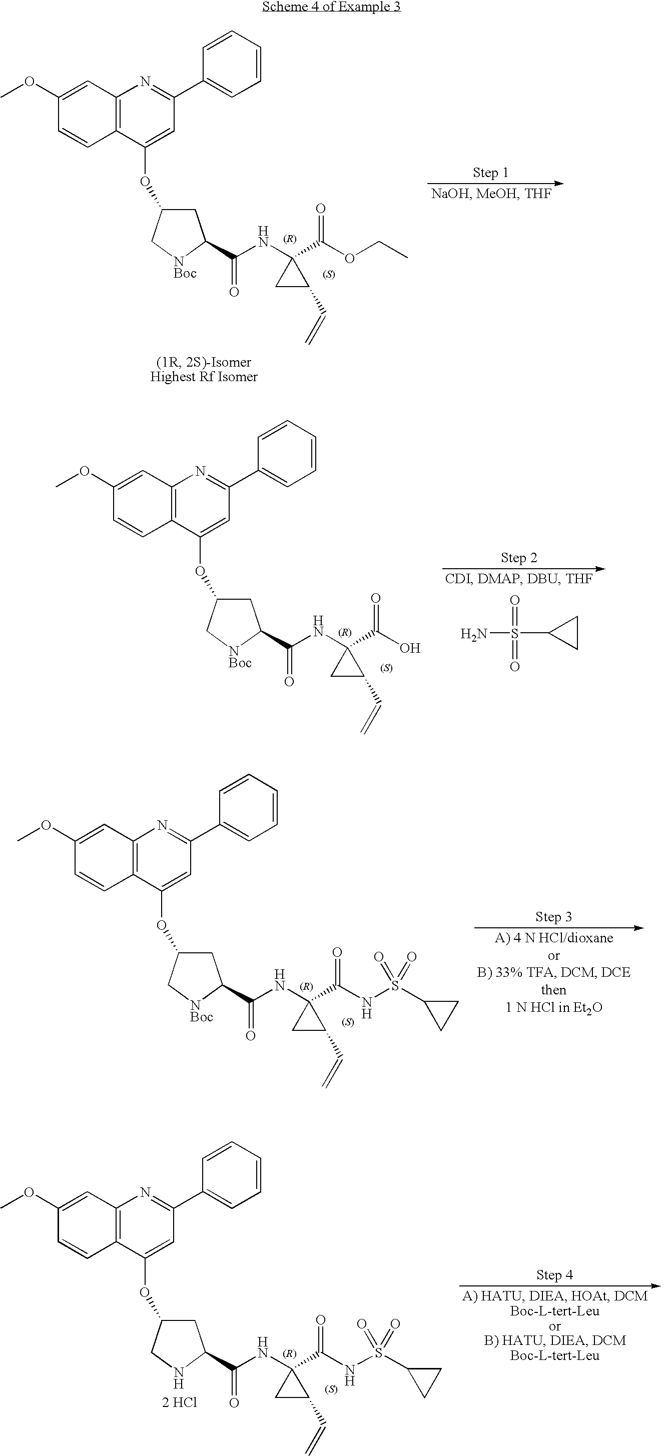

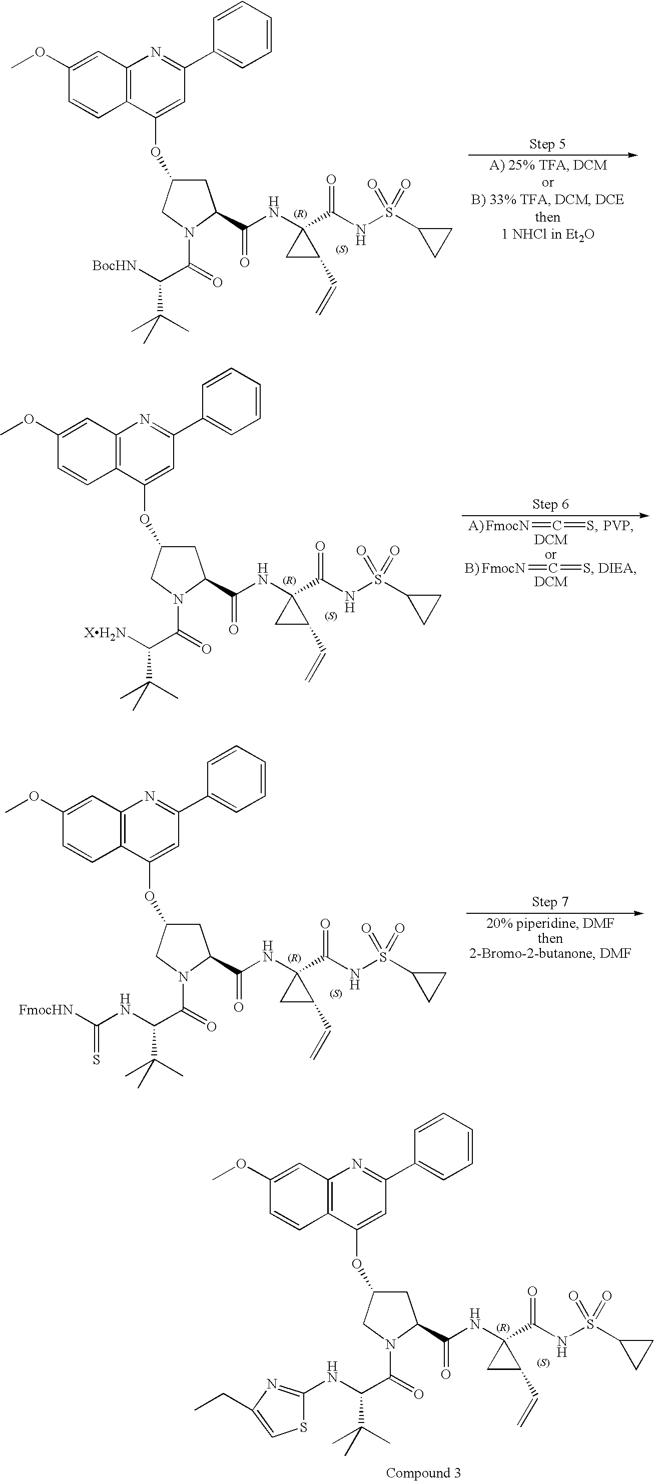

### Step 1:

The (1R, 2S) P1 isomer of Step 2, scheme 3 (9.86 g, 16.4 mmol) was treated with 1N NaOH (50 mL, 50 mmol) in a mixture of THF (150 mL) and methanol (80 mL) for 12 hours. The mixture was concentrated in vacuo until only the aqueous phase remained. Water (100 mL) was added and 1N HCl was added slowly until pH 3 was achieved. The mixture was then extracted with ethyl acetate (3×200 mL), and the combined organic extracts were washed with brine, dried over anhydrous sodium sulfate, and filtered. The filtrate was concentrated in vacuo to give the desired product as a white powder (9.2 g, 98% yield). ¹H NMR (CD₃OD) δ 1.41 (s, 2H), 1.45 (s, 9H), 1.77 (dd, J=7.9, 5.5 Hz, 1H), 2.16-2.21 (m, 1H), 2.44-2.51 (m, 1H), 2.74-2.79 (m, 1H), 3.93-3.96 (m, 2H), 3.98 (s, 3H), 4.44 (t, J=7.9 Hz, 1H), 5.11 (d, J=9.5 Hz, 1H), 5.30 (d, J=17.1 Hz, 1H), 5.52 (s, 1H), 5.79-5.86 (m, 1H), 7.22 (dd, J=9.16, 2.14 Hz, 1H), 7.32 (s, 1H), 7.43 (d, J=2.14 Hz, 1H), 7.54-7.60 (m, 3H), 8.04 (dd, J=7.8, 1.4 Hz, 2H), 8.08 (d, J=9.1 Hz, 1H); LC-MS (MS m/z 574 (M⁺+1). (M⁺+1).

### Step 2:

The product of Step 1 (7.54 g, 13.14 mmol) was combined with CDI (3.19 g, 19.7 mmol) and DMAP (2.41 g, 19.7 mmol) in anhydrous THF, and the resulting mixture was heated to reflux for 45 minutes. The slightly opaque mixture was allowed to cool to room temperature, and to it was added cyclopropylsulfonamide (1.91 g, 15.8 g). Upon addition of DBU (5.9 mL, 39.4 mmol), the mixture became clear. The brown solution was stirred overnight. The mixture was then concentrated in vacuo to an oil and was redissolved in ethyl acetate (500 mL). The solution was washed with pH 4 buffer (3×200 mL), and the combined buffer washes were back-extracted with ethyl acetate (200 mL). The combined organics were washed with brine (150 mL) and dried over anhydrous sodium sulfate and filtered. Concentration of the filtrate in vacuao gave a beige solid. The crude product was purified by flash chromatography (SiO₂, eluted with 25% hexanes/ethyl acetate) to give the desired product (5.85 g, 66% yield). ¹H NMR (CD₃OD) δ 1.03-1.09 (m, 2H), 1.15-1.28 (m, 2H), 1.40-1.44 (m, 2H), 1.46 (s, 9H), 1.87 (dd, J=8.1, 5.6 Hz, 1H), 2.21-2.27 (m, 1H), 2.36-2.42 (m, 1H), 2.65 (dd, J=13.7, 6.7 Hz, 1H), 2.93-2.97 (m, 1H), 3.90-3.96 (m, 2H), 4.00 (s, 3H), 4.40 (dd, J=9.5, 7.0 Hz, 1H), 5.12 (d, J=10.4 Hz, 1H), 5.31 (d, J=17.4 Hz, 1H), 5.64 (s, 1H), 5.73-5.80 (m, 1H), 7.30 (dd, J=9.2, 2.1 Hz, 1H), 7.40 (s, 1H), 7.47 (s, 1H), 7.61-7.63 (m, 3H), 8.04-8.05 (m, 2H), 8.15 (d, J=9.5 Hz, 1H); LC-MS (MS m/z 677 (M⁺+1).

### Step 3A:

The product of Step 2 (5.78 g, 8.54 mmol) was treated with 4.0M HCl in 1,4-dioxane (50 mL, 200 mmol) overnight. The reaction mixture was concentrated in vacuo and placed in a vacuum oven at 50° C. for several days. The desired product was obtained as a beige powder (5.85 g, quantitative). ¹H NMR (methanol-d₄) δ 1.03-1.18 (m, 3H), 1.26-1.30 (m, 1H), 1.36-1.40 (m, 2H), 1.95 (dd, J=8.2, 5.8 Hz, 1H), 2.37 (q, J=8.9 Hz, 1H), 2.51-2.57 (m, 1H), 2.94-2.98 (m, 1H), 3.09 (dd, J=14.6, 7.3 Hz, 1H), 3.98 (d, J=3.7 Hz, 1H), 3.99 (s, 1H), 4.08 (s, 3H), 4.80 (dd, J=10.7, 7.6 Hz, 1H), 5.15 (dd, J=10.2, 1.4 Hz, 1H), 5.32 (dd, J=17.1, 1.2 Hz, 1H), 5.61-5.69 (m, 1H), 5.99 (t, J=3.7 Hz, 1H), 7.51 (dd, J=9.3, 2.3 Hz, 1H), 7.59 (d, J=2.4 Hz, 1H), 7.65 (s, 1H), 7.72-7.79 (m, 3H), 8.09 (dd, J=7.0, 1.5 Hz, 2H), 8.53 (d, J=9.2 Hz, 1H); LC-MS (MS m/z 577 (M⁺+1).

### Step 3B:

To a solution of (2S, 4R)-tert-butyl 2-((1R, 2S)-1-(cyclopropylsulfonylcarbanoyl)-2-vinylcyclopropylcarbamoyl)-4-(7-methoxy-2-phenylquinolin-4-yloxy)pyrrolidine-1-carboxylate, the product of step 2 (3.0 g, 4.43 mmol) in 1:1 DCM (25 mL)/DCE (25.00 mL) was added trifluoroacetic acid (25 mL, 324 mmol). After stirring at 25° C. for 0.5 h, the resulting brown reaction mixture was concentrated to brown vicous oil which was redissolved in DCE (50 mL) and reconcentrated. The residue was dissolved in DCM (10 mL) and was added dropwise to a solution of 1N HCl in Et₂O (50 mL, 50.0 mmol). The resulting light brown precipitate was filtered, washed with a solution of 1N HCl in Et₂O (40 mL) and dried in a 50° C. vacuum oven for 1 h to afford (2S, 4R)-N-((1R, 2S)-1-(cyclopropylsulfonylcarbanoyl)-2-vinylcyclopropyl)-4-(7-methoxy-2-phenylquinolin-4-yloxy)pyrrolidine-2-carboxamide, 2 HCl salt (2.8 g, 4.31 mmol, 97% yield) as a light brown solid. ¹H-NMR showed the product contained about 0.75 equivalents of tetramethyl urea byproduct (signal at 2.83 ppm as a siglet), but this material was used without further purification in the next step. ¹H NMR (500 MHz, MeOD) δ ppm 1.0-1.2 (m, 3H), 1.2-1.3 (m, 1H), 1.4 (dd, J=9.5, 5.5 Hz, 2H), 1.9 (dd, J=7.9, 5.8 Hz, 2H), 2.4 (q, J=8.7 Hz, 1H), 2.5-2.6 (m, 1H), 2.9-2.9 (m, 1H), 3.1 (dd, J=14.6, 7.3 Hz, 1H), 4.0-4.0 (m, 2H), 4.1 (s, 3H), 4.8-4.9 (m, 1H), 5.1 (dd, J=10.4, 1.5 Hz, 1H), 5.3 (dd, J=17.2, 1.4 Hz, 1H), 5.6-5.7 (m, 1H), 6.0 (s, 1H), 7.5 (dd, J=9.3, 2.3 Hz, 1H), 7.6 (d, J=2.4 Hz, 1H), 7.7 (s, 1H), 7.7-7.8 (m, 3H), 8.1 (d, J=6.7 Hz, 2H), 8.6 (d, J=9.2 Hz, 1H). LC-MS, MS m/z 577.2 (M⁺+H).

Step 4A:

To a solution of the product from step 3A (0.671 mmol) in DCM (10 mL) was added DIEA (542 μL, 3.36 mmol), HATU (354 mg, 1.01 mmol), HOAt (127 mg, 1.01 mmol), and Boc-L-Tle-OH (173 mg, 0.805 mmol). After stirring at rt for 16 h, the solvent was concentrated and the resulting brown viscous oil was purified by flash column chromatography (SiO₂, eluted with 95% MeOH in DCM) to give a slightly yellow foam (527 mg, 99% yield). LC-MS (MS m/z 790 (M⁺+1)).

### Step 4B:

To a solution of (2S, 4R)-N-((1R, 2S)-1-(cyclopropylsulfonylcarbamoyl)-2-vinylcyclopropyl)-4-(7-methoxy-2-phenylquinolin-4-yloxy)pyrrolidine-2-carboxamide, 2 HCl salt, the product of step 3B (1.2 g, 1.847 mmol), N,N-diisopropylethylamine (1.126 mL, 6.47 mmol) and Boc-L-Tle-OH (0.513 g, 2.217 mmol) in DCM (15 mL) was added HATU (1.054 g, 2.77 mmol). The resulting light brown reaction mixture was stirred at rt for 13 h, the reaction mixture was concentrated and re-dissolved in EtOAc (50 mL) and washed with 1N aqueous HCl (25 mL). The acidic aqueous layer was extracted with EtOAc (50 mL). The organic layers were combined and washed with 10% aqueous Na₂CO₃ (20 mL), brine, dried over MgSO₄ and concentrated. The resulting vicous brown oil was purified by flash column chromatography (SiO₂, eluted with 95:5 DCM:MeOH) to give tert-butyl (S)-1-((2S, 4R)-2-((1R,2S)-1-(cyclopropylsulfonylcarbamoyl)-2-vinylcyclopropylcarbamoyl)-4-(7-methoxy-2-phenylquinolin-4-yloxy)pyrrolidin-1-yl)-3,3-dimethyl-1-oxobutan-2-ylcarbamate a a light brown foam which was of sufficient purity for use in the next step. However, for the analytical sample for 25 characterization by NMR, 85 mg of this product was further purified by reverse phase HPLC using solvent system and conditions as the following: solvent A=H₂O, solvent B=MeOH, both containing 0.1% TFA; 50% B to 100% B 20 mins, hold at 100% B 4 mins. The combined HPLC fractions was neutralized with 1N aqueous NaOH and concentrated until mostly water remained. The resulting white creamy mixture was extracted with EtOAc (2×25 mL). The organic layers were combined, washed with brine, dried over MgSO4, concentrated and dried in vacuo to afford analytically pure white powder product. ¹H NMR (500 MHz, MeOD) δ ppm 0.9-1.0 (m, 2H), 1.0 (s, 9H), 1.1-1.2 (m, 1H), 1.2-1.2 (m, 3H), 1.3 (s, 9H), 1.4-1.4 (m, 1H), 1.9 (dd, J=7.9, 5.5 Hz, 1H), 2.2 (q, J=8.7 Hz, 1H), 2.3-2.3 (m, 1H), 2.6 (dd, J=13.9, 6.9 Hz, 1H), 2.9-3.0 (m, 1H), 3.9 (s, 3H), 4.0-4.1 (m, 1H), 4.2 (d, J=9.5 Hz, 1H), 4.5-4.5 (m, 2H), 5.1 (d, J=11.0 Hz, 1H), 5.3 (d, J=17.1 Hz, 1H), 5.5 (s, 1H), 5.7-5.8 (m, 1H), 6.6 (d, J=9.5 Hz, 1H), 7.1 (dd, J=9.0, 1.7 Hz, 1H), 7.2 (s, 1H), 7.4 (d, J=1.8 Hz, 1H), 7.5-7.5 (m, 3H), 8.0 (t, J=7.3 Hz, 3H). ¹³C NMR (126 MHz, MeOD) δ ppm 5.6, 5.8, 17.6, 22.6, 26.1, 27.6, 31.2, 34.7, 35.0, 35.2, 41.7, 42.8, 54.4, 55.1, 59.5, 59.9, 77.2, 79.5, 99.2, 106.4, 115.5, 117.6, 117.9, 118.4, 123.3, 128.0, 128.8, 129.7, 133.3, 140.1, 151.0, 151.1, 157.1, 160.2, 161.0, 162.3, 169.8, 172.5, 174.0. LC-MS, MS m/z 790.30 (M⁺+H).

### Step 5A:

A solution of the product from step 4A (950 mg, 1.20 mmol) in DCM (75 mL) was treated with TFA (25 mL) slowly to control CO₂ gas from vigorously bubbling. After stirring at rt for 1.5 hr, the solvent was concentrated to give a light brown slurry and Et₂O was added to effect a precipitation. The light brown product (1.10 g, 99% yield) bis TFA salt was obtained by a vacuum filtration and used without further purification. LC-MS (MS m/z 690 (M⁺+1)).

### Step 5B:

To a solution of tert-butyl (S)-1-((2S, 4R)-2-((1R, 2S)-1-(cyclopropylsulfonylcarbamoyl)-2-vinylcyclopropylcarbamoyl)-4-(7-methoxy-2-phenylquinolin-4-yloxy)pyrrolidin-1-yl)-3,3-dimethyl-1-oxobutan-2-ylcarbamate, the product of step 4B (1.00 g, 1.266 mmol) in 1:1 DCM (5 mL) and DCE (5.00 mL) was added trifluoroacetic acid (5 mL, 64.9 mmol). After stirring at 25° C. for 15 mins, the reaction mixture was concentrated. The resulting viscous brown oil was redissolved in DCM (3 mL) and was added dropwise to a vigorously stirred solution of 1N HCl (50 mL) in Et₂O. The resulting light brown precipitate was filtered, washed with Et₂O (25 mL) and dried in a 50° C. vacuum oven for 2 h to afford (2S, 4R)-1-((S)-2-amino-3,3-dimethylbutanoyl)-N-((1R, 2S)-1-(cyclopropylsulfonylcarbamoyl)-2-vinylcyclopropyl)-4-(7-methoxy-2-phenqinolin-4-yloxy)pyrrolidine-2-carboxamide, 2 HCl salt (0.907 g, 1.189 mmol, 94% yield) as a light brown solid which was of sufficient purity for use in the next step. However, for the analytical sample for characterization by NMR, 80 mg of product was further purified by reverse phase HPLC using solvent system and conditions as the following: solvent A=H2O, solvent B=MeOH, both containing 0.1% TFA; 15% B to 100% B 20 mins, hold at 100% B 4 mins. The combined HPLC fractions was treated with 1N aqueous HCl (3 mL), concentrated to dryness and dried in vacuo to afford the bis-HCl salt product as white powder. ¹H NMR (500 MHz, MeOD) δ ppm 1.0-1.1 (m, 4H), 1.2 (s, 9H), 1.2-1.3 (m, 2H), 1.4 (s, 1H), 1.9 (s, 1H), 2.3 (d, J=5.8 Hz, 1H), 2.4 (s, 1H), 2.8-2.9 (m, 1H), 2.9-3.0 (m, 1H), 4.1 (s, 3H), 4.2 (s, 2H), 4.6 (d, J=8.2 Hz, 1H), 4.8 (s, 1H), 5.1 (d, J=10.4 Hz, 1H), 5.3 (d, J=17.1 Hz, 1H), 5.6-5.7 (m, 1H) 5.9 (s, 1H), 7.5 (d, J=8.2 Hz, 1H), 7.6-7.7 (m, 2H), 7.7-7.8 (m, 3H), 8.1 (d, J=4.0 Hz, 2H), 8.5 (d, J=8.5 Hz, 1H). ¹³C NMR (MeOD) δ ppm 5.0 (s), 5.8, 5.8, 22.4, 25.9, 31.3, 34.6, 34.9, 35.0, 41.8, 42.8, 54.7, 56.1, 59.5, 60.5, 80.4, 99.8, 101.5, 166.8, 168.2, 169.4, 173.2. LC-MS, MS m/z 690.2 (M++H).

### Step 6A:

To a solution of product of step 5A (0.132 g, 0.143 mmol) in DCM (2 mL) was added polyvinylpyridine (PVP) (0.046 g, 0.429 mmol) and Fmoc-isothiocyanate (0.042 g, 0.150 mmol). The resulting brown solution was stirred at rt. After 16 hr, solvent was removed and residue was purified by flash column chromatography (SiO₂, eluted with 95:5 DCM:MeOH) to give a light brown solid product (0.126 mg, 91% yield).

### Step 6B:

To a solution of (2S, 4R)-1-((S)-2-amino-3,3-dimethylbutanoyl)-N-((1R, 2S)-1-(cyclopropylsulfonylcarbamoyl)-2-vinylcyclopropyl)-4-(7-methoxy-2-phenylquinolin-4-yloxy)pyrrolidine-2-carboxamide, 2 HCl, the product of step 5B (0.500 g, 0.656 mmol) and N,N-diisopropylethylamine (0.343 mL, 1.967 mmol) in DCM (8 mL) was added Emoc-isothiocyanate (0.240 g, 0.852 mmol). The resulting brown reaction mixture was stirred at 25° C. for 16 h. The reaction mixture was concentrated, the residue was taken up with EtOAc (50 mL) and washed with 0.1N aqueous HCl (10 mL). The aqueous layer was extracted with EtOAc (25 mL). The organic layers were combined, washed with brine, dried over MgSO₄ and concentrated to a yellow solid crude product which was purified by flash column chromatography (SiO₂, eluted with 95:5 DCM:MeOH) to afford (2S, 4R)-1-((S)-2-(3-(((9H-fluoren-9-yl)methoxy)carbonyl)thioureido)-3,3-dimethylbutanoyl)-N-((1R,2S)-1-(cyclopropylsulfonylcarbamoyl)-2-vinylcyclopropyl)-4-(7-methoxy-2-phenylquinolin-4-yloxy)pyrrolidine-2-carboxamide (615.4 mg, 0.634 mmol, 97% yield) as a light yellow solid which was of sufficient purity for use in the next step. However, 45 mg of product was further purified by reverse phase HPLC using solvent system and conditions as the following: solvent A=H2O, solvent B=MeOH, both containing 0.1% TPA; 50% B to 100% B 20 mins, hold at 100% B 4 mins. Note: a half mL of DMF and 1 mL of MEOH were used to dissolve the HPLC sample in order to prevent sample precipitation on the HPLC column. After concentration of the combined HPLC fractions until mostly water remained, 1N aqueous NaOH was added to neutralize the white creamy mixture and it was then extracted with EtOAc (2×25 mL). The organic layers were combined, dried over MgSO₄ and concentrated to afford the an alytically pure sample as a white powder which was used for LC/MS and NMR analysis. ¹H NMR (500 MHz, MeOD) δ ppm 0.1-1.0 (m, 2H), 1.1 (s, 9H), 1.2-1.2 (m, 2H), 1.2 (t, J=7.2 Hz, 1H), 1.3 (s, 1H), 1.4 (dd, J=9.3, 5.3 Hz, 1H), 1.9 (dd, J=8.1, 5.6 Hz, 1H), 2.0 (s, 1H), 2.2 (q, J=8.7 Hz, 1H), 2.4-2.4 (m, 1H), 2.7 (dd, J=14.2, 6.9 Hz, 1H), 2.9-2.9 (m, 1H), 4.0 (s, 3H), 4.1-4.1 (m, 1H), 4.2 (t, J=6.9 Hz, 1H), 4.4-4.5 (m, 2H), 4.6 (dd, J=10.7, 7.0 Hz, 1H), 4.8 (d, J=7.3 Hz, 1H), 5.0 (d, J=12.2 Hz, 1H), 5.1 (dd, J=10.4, 1.2 Hz, 1H), 5.3 (dd, J=17.2, 1.1 Hz, 1H), 5.6-5,7 (m, 1H), 5.8 (s, 1H), 7.3-7.3 (m, 3H), 7.4 (t, J=7.5 Hz, 2H), 7.4 (d, J=2.1 Hz, 1H), 7.5 (s, 1H), 7.6 (d, J=7.0 Hz, 2H), 7.6-7.7 (m, 3H), 7.8 (d, J=7.6 Hz, 2H), 8.0 (dd, J=7.6, 1.8 Hz, 2H), 8.2 (d, J=9.2 Hz, 1H), 10.3 (d, J=7.3 Hz, 1H).

^{—}C NMR (MeOD) δ ppm 5.6, 5.6, 13.5, 22.0, 26.3, 31.2, 34.7, 34.8, 35,4, 42.0, 42.8, 47.0, 54.2, 55.8, 60.0, 60.5, 64.3, 4.4, 68.1, 79.8, 100.9, 101.3, 115.3, 117.7, 120.0, 120.2, 125.1, 125.2, 127.3, 128.0, 128.7, 129.6, 132.1, 133.2, 141.6, 143.6, 143.8, 144.7, 154.1, 157.9, 164.8, 165.5, 169.4, 170.6, 172.0, 174.1, 180.8, 180.9, 188.0. LC-MS, MS m/z 971.18 (M++H).

### Step 7:

To a solution of product of step 6 (0.342 mg, 0.352 mmol) in DMF (4 mL) was added piperidine (0.805 mL). The resulting brown solution mixture was stirred at rt overnight. Solvent and excess piperidine were removed using a roto-evaporator under reduced pressure to give the desired product and also an equivalent of 1-((9H-fluoren-9-yl)methyl)piperidine byproduct. The resulting crude product mixture was used in the next step without further purification. LC-MS, MS m/z 749 (M⁺+H). To a solution of the residue from above (77.3 mg, 0.076 mmol) in DMF (2 mL) was added 2-bromo-2-butanonone (23.0 mg, 0.152 mmol). After stirring at rt for 16 hr, the reaction mixture was concentrated and product was purified by column chromatography to give compound 3. LC-MS, MS m/z 801.31 (M⁺+H).

### EXAMPLE 4

### Preparation of Compound 4

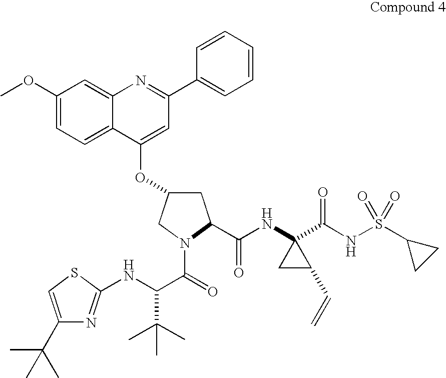

Compound 4 was prepared by the same procedure as described for the preparation of the product of compound 3, except 1-bromopinacolone was used instead of 2-bromo-2-butanonone. LC-MS, MS m/z 829.38 (M⁺+H).

### EXAMPLE 5

### Preparation of Compound 5

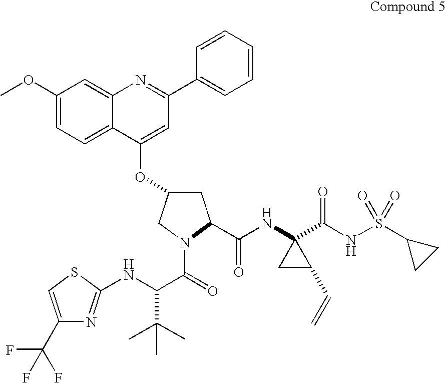

Compound 5 was prepared by the same procedure as described for the preparation of compound 3, except 1-bromo-1,1,1-trifluoropropanone was used instead of 2-bromo-2-butanonone. LC-MS, MS m/z 841.28 (Me⁺+H).

### Biological Studies

HCV NS3/4A protease complex enzyme assays and cell-based HCV replicon assays were utilized in the present disclosure, and were prepared, conducted and validated as follows:

### Generation of Recombinant HCV NS3/4Protease Complex

HCV NS3 protease complexes, derived from the BMS strain, H77 strain or J4L6S strain, were generated, as described below. These purified recombinant proteins were generated for use in a homogeneous assay (see below) to provide an indication of how effective compounds of the present disclosure would be in inhibiting HCV NS3 proteolytic activity.

Serum from an HCV-infected patient was obtained from Dr. T. Wright, San Francisco Hospital. An engineered full-length cDNA (compliment deoxyribonucleic acid) template of the HCV genome (BMS strain) was constructed from DNA fragments obtained by reverse transcription-PCR (RT-PCR) of serum RNA (ribonucleic acid) and using primers selected on the basis of homology between other genotype 1a strains. From the determination of the entire genome sequence, a genotype 1a was assigned to the HCV isolate according to the classification of Simmonds et al. (See P Simmonds, K A Rose, S Graham, S W Chan, F McOmish, B C Dow, E A Follett, P L Yap and H Marsdeni, J. Clin. Microbiol., 31(6):1493-1503 (1993)). The amino acid sequence of the nonstructural region, NS2-5B, was shown to be >97% identical to HCV genotype 1a (H77) and 87% identical to genotype 1b (J4L6S). The infectious clones, H77 (1a genotype) and J4L6S (1b genotype) were obtained from R. Purcell (NIH) and the sequences are published in Genbank (AAB67036, see Yanagi, M., Purcell, R. H., Emerson, S. U. and Bukh, J. Proc. Natl. Acad. Sci. U.S.A. 94(16):8738-8743 (1997); AF054247, see Yanagi, M., St Claire, M., Shapiro, M., Emerson, S. U., Purcell, R. H. and Bukh, J, Virology 244 (1), 161-172. (1998)).

The H77 and J4L6S strains were used for production of recombinant NS3/4A protease complexes. DNA encoding the recombinant HCV NS3/4A protease complex (amino acids 1027 to 1711) for these strains were manipulated as described by P. Gallinari et al. (see Gallinari P, Paolini C, Breruian D, Nardi C, Steinlkuhler C, De Francesco R. Biochemistry. 38(17);5620-32, (1999)). Briefly, a three-lysine solubilizing tail was added at the 3′-end of the NS4A coding region. The cysteine in the P1 position of the NS4A-NS4B cleavage site (amino acid 1711) was changed to a glycine to avoid the proteolytic cleavage of the lysine tag. Furthermore, a cysteine to serine mutation was introduced by PCR at amino acid position 1454 to prevent the autolytic cleavage in the NS3 helicase domain. The variant DNA fragment was cloned in the pET21b bacterial expression vector (Novagen) and the NS3/4A complex was expressed in Escherichia. coli strain BL21 (DE3) (Invitrogen) following the protocol described by P. Gallinari et al. (see Gallinari P, Breinan D, Nardi C, Brunetti M, Tomei L, Steinkuhler C, De Francesco R., J Virol. 72(8):6758-69 (1998)) with modifications. Briefly, the NS3/4A protease complex expression was induced with 0.5 millimolar (mM) Isopropyl β-D-1-thiogalactopyranoside (IPTG) for 22 hours (h) at 20° C. A typical fermentation (1 Liter (L)) yielded approximately 10 grams (g) of wet cell paste. The cells were resuspended in lysis buffer (10 mL/g) consisting of 25 mM N-(2-Hydroxyethyl)Piperazine-N′-(2-Ethane Sulfonic acid) (HEPES), pH 7.5, 20% glycerol, 500 mM Sodium Chloride (NaCl), 0.5% Triton X-100, 1 microgram/milliliter (“μg/mL”) lysozyme, 5 mM Magnesium Chloride (MgCl₂), 1 μg/ml DnaseI, 5mM β-Mercaptoethanol (βME), Protease inhibitor-Ethylenediamine Tetraacetic acid (EDTA) free (Roche), homogenized and incubated for 20 minutes (min) at 4° C. The homogenate was sonicated and clarified by ultra-centrifugation at 235000 g for 1 hour (h) at 4° C. Imidazole was added to the supernatant to a final concentration of 15 mM and the pH adjusted to 8.0. The crude protein extract was loaded on a Nickel-Nitrilotriacetic acid (Ni-NTA) column pre-equilibrated with buffer B (25 mM HEPES, pH 8.0, 20% glycerol, 500 mM NaCl, 0.5% Triton X-100, 15 mM imidazole, 5 mM βMB). The sample was loaded at a flow rate of 1 mL/min. The column was washed with 15 column volumes of buffer C (same as buffer B except with 0.2% Triton X-100). The protein was eluted with 5 column volumes of buffer D (same as buffer C except with 200 mM Imidazole).

NS3/4A protease complex-containing fractions were pooled and loaded on a desalting column Superdex-S200 pre-equilibrated with buffer D (25 mM HEPES, pH 7.5, 20% glycerol, 300 mM NaCl, 0.2% Triton X-100, 10 mM βME). Sample was loaded at a flow rate of 1 mL/min. NS3/4A protease complex-containing fractions were pooled and concentrated to approximately 0.5 mg/ml. The purity of the NS3/4A protease complexes, derived from the BMS, H77 and J4L6S strains, were judged to be greater than 90% by SDS-PAGE and mass spectrometry analyses. The enzyme was stored at −80° C., thawed on ice and diluted prior to use in assay buffer.

### FRET Peptide Assay to Monitor HCV NS3/4A Proteolytic Activity

The purpose of this in vitro assay was to measure the inhibition of HCV NS3 protease complexes, derived from the BMS strain, H77 strain or J4L6S strain, as described above, by compounds of the present disclosure. This assay provides an indication of how effective compounds of the present disclosure would be in inhibiting HCV NS3 proteolytic activity.

In order to monitor HCY NS3/4A protease activity, an NS3/4A peptide substrate was used. The substrate was RET S1 (Resonance Energy Transfer Depsipeptide Substrate; AnaSpec, Inc. cat #22991)(FRET peptide), described by Taliani et al. in Anal. Biochem. 240(2):60-67 (1996). The sequence of this peptide is loosely based on the NS4A/NS4B natural cleavage site for the HCV NS3 protease except there is an ester linkage rather than an amide bond at the cleavage site. The peptide also contains a fluorescence donor, EDANS, near one end of the peptide and an acceptor, DABCYL, near the other end. The fluorescence of the peptide is quenched by intermolecular resonance energy transfer (RET) between the donor and the acceptor, but as the NS3 protease cleaves the peptide the products are released from RET quenching and the fluorescence of the donor becomes apparent.

The peptide substrate was incubated with one of the three recombinant NS3/4A protease complexes, in the absence or presence of a compound of the present disclosure. The inhibitory effects of a compound were determined by monitoring the formation of fluorescent reaction product in real time using a Cytofluor Series 4000.

The reagents were as follow: HEPES and Glycerol (Ultrapure) were obtained from GIBCO-BRL. Dimethyl Sulfoxide (DMSO) was obtained from Sigma. β-Mercaptoethanol was obtained from Bio Rad.

Assay buffer: 50 mM HEPES, pH 7.5; 0.15 M NaCl; 0.1% Triton; 15% Glycerol; 10 mM βME. Substrate: 2 μM final concentration (from a 2 mM stock solution in DMSO stored at −20° C.). HCO NS3/4A protease type 1a (1b), 2-3 nM final concentration (from a 5 μM stock solution in 25 mM HEPES, pH 7.5, 20% glycerol, 300 mM NaCl, 0.2% Triton-X100, 10 mM βME). For compounds with potencies approaching the assay limit, the assay was made more sensitive by adding 50 μg/ml Bovine Serum Albumin (Sigma) to the assay buffer and reducing the end protease concentration to 300 pM.

The assay was performed in a 96-well polystyrene black plate from Falcon. Each well contained 25 μl NS3/4A protease complex in assay buffer, 50 μl of a compound of the present disclosure in 10% DMSO/assay buffer and 25 μl substrate in assay buffer. A control (no compound) was also prepared on the same assay plate. The enzyme complex was mixed with compound or control solution for 1 min before initiating the enzymatic reaction by the addition of substrate. The assay plate was read immediately using the Cytofluor Series 4000 (Perspective Biosystems). The instrument was set to read an emission of 340 nm and excitation of 490 nm at 25° C. Reactions were generally followed for approximately 15 min.

The percent inhibition was calculated with the following equation:

100−[(δFᵢₙₕ/δF_{con})×100]

where δF is the change in fluorescence over the linear range of the curve. A non-linear curve fit was applied to the inhibition-concentration data, and the 50% effective concentration (IC₅₀) was calculated by the use of Excel XLfit software using the equation, y=A+((B−A)/(1+((C/×)̂D))).

All of the compounds tested were found to inhibit the activity of the NS3/4A protease complex with IC50's of 135 nM or less. Further, compounds of the present disclosure, which were tested against more than one type of NS3/4A complex, were found to have similar inhibitory properties though the compounds uniformly demonstrated greater potency against the 1b strains as compared to the 1a strains.

### Specificity Assays

The specificity assays were performed to demonstrate the in vitro selectivity of the compounds of the present disclosure in inhibiting HCV NS3/4A protease complex as compared to other serine or cysteine proteases.

The specificities of compounds of the present disclosure were determined against a variety of serine proteases: human neutrophil elastase (HNE), porcine pancreatic elastase (PPE) and human pancreatic chymotrypsin and one cysteine protease: human liver cathepsin B. In all cases a 96-well plate format protocol using a fluorometric Amino-Methyl-Coumarin (AMC) substrate specific for each enzyme was used as described previously (PCT Patent Application No. WO 00/09543) with some modifications to the serine protease assays. All enzymes were purchased from Sigma, EMDbiosciences while the substrates were from Bachem, Sigma and EMDbiosciences.

Compound concentrations varied from 100 to 0.4 μM depending on their potency. The enzyme assays were each initiated by addition of substrate to enzyme-inhibitor pre-incubated for 10 min at room temperature and hydrolysis to 15% conversion as measured on cytofluor.

The final conditions for each assay were as follows:
50 mM Tris(hydroxymethyl)aminomethanae hydrochloride (Tris-HCl) pH 8, 0.5 M Sodium Sulfate (Na₂SO₄), 50 mM NaCl, 0.1 mM EDTA, 3% DMSO, 0.01% Tween-20 with 5 μM LLVY-AMC and 1 nM Chynotrypsin.
50 mM Tris-HCl, pH 8.0, 50 mM NaCl, 0.1 mM EDTA, 3% DMSO, 0.02% Tween-20, 5 μM succ-AAPV-AMC and 20 nM HNE or 8 nM PPE.
100 mM NaOAC (Sodium Acetate) pH 5.5, 3% DMSO, 1 mM TCEP (Tris(2-carboxyethyl)phosphine hydrochloride), 5 nM Cathepsin B (enzyme stock activated in buffer containing 20 mM TCEP before use), and 2 μM Z-FR-AMC diluted in H₂O.

The percentage of inhibition was calculated using the formula:

[1−((UVᵢₙₕ−UV_{blank})/(UV_{ctl}−UV_{blank}))]×100

A non-linear curve fit was applied to the inhibition-concentration data, and the 50% effective concentration (IC₅₀) was calculated by the use of Excel XLfit software.

### Generation of HCV Replicon

An HCV replicon whole cell system was established as described by Lohmann V, Korner F, Koch J, Herian U, Theilmann L, Bartenschlager R., Science 285(5424):110-3 (1999). This system enabled us to evaluate the effects of our HCV Protease compounds on HCV RNA replication. Briefly, using the HCV strain 1b sequence described in the Lohmann paper (Assession number: AJ238799), an HCV cDNA was synthesized by Operon Technologies, Inc. (Alameda, Calif.), and the full-length replicon was then assembled in plasmid pGem9zf(+) (Promega, Madison, Wis.) using standard molecular biology techniques. The replicon consists of (i) the HCV 5′ UTR fused to the first 12 amino acids of the capsid protein, (ii) the neomycin phosphotransferase gene (neo), (iii) the IRES from encephalomyocarditis virus (EMCV), and (iv) HCV NS3 to NS5B genes and the HCV 3′ UTR. Plasmid DNAs were linearized with ScaI and RNA transcripts were synthesized in vitro using the T7 MegaScript transcription kit (Aibion, Austin, Tex.) according to manufacturer's directions. In vitro transcripts of the cDNA were transfected into the human hepatoma cell line, HUH-7. Selection for cells constitutively expressing the HCV replicon was achieved in the presence of the selectable marker, neomycin (G418). Resulting cell lines were characterized for positive and negative strand RNA production and protein production over time.

### HCV Replicon FRET Assay

The HCV replicon FRET assay was developed to monitor the inhibitory effects of compounds described in the disclosure on NCV viral replication. HUH-7 cells, constitutively expressing the HCV replicon, were grown in Dulbecco's Modified Eagle Media (DMEM) (Gibco-BRL) containing 10% Fetal calf serum (FCS) (Sigma) and 1 mg/ml G418 (Gibco-BRL). Cells were seeded the night before (1.5×10⁴ cells/well) in 96-well tissue-culture sterile plates. Compound and no compound controls were prepared in DMEM containing 4% FCS, 1:100 Penicillin/Streptomysin (Gibco-BRL), 1:100 L-glutamine and 5% DMSO in the dilution plate (0.5% DMSO final concentration in the assay). Compound/DMSO mixes were added to the cells and incubated for 4 days at 37° C. After 4 days, cells were first assessed for cytotoxicity using alamar Blue (Trek Diagnotstic Systems) for a CC₅₀ reading. The toxicity of compound (CC₅₀) was determined by adding 1/10^{th} volume of alamar Blue to the media incubating the cells. After 4 h, the fluorescence signal from each well was read, with an excitation wavelength at 530 nm and an emission wavelength of 580 nm, using the Cytofluor Series 4000 (Perspective Biosystems). Plates were then rinsed thoroughly with Phosphate-Buffered Saline (PBS) (3 times 150 μl). The cells were lysed with 25 μl of a lysis assay reagent containing an HCV protease substrate (5× cell Luciferase cell culture lysis reagent (Prornega #E153A) diluted to 1× with distilled water, NaCl added to 150 mM final, the FRET peptide substrate (as described for the enzyme assay above) diluted to 10 μM final from a 2 mM stock in 100% DMSO. The plate was then placed into the Cytofluor 4000 instrument which had been set to 340 nm excitation/490 nm emission, automatic mode for 21 cycles and the plate read in a kinetic mode. EC₅₀ determinations were carried out as described for the IC₅₀ determinations.

### HCV Replicon Luciferase Reporter Assay

As a secondary assay, EC₅₀ determinations from the replicon FRET assay were confirmed in a replicon luciferase reporter assay. Utilization of a replicon luciferase reporter assay was first described by Krieger et al (Krieger N, Lohmann V, and Bartenschlager R, J. Virol. 75(10):4614-4624 (2001)). The replicon construct described for our FRET assay was modified by inserting cDNA encoding a humanized form of the Renilla luciferase gene and a linker sequence fused directly to the 3′-end of the luciferase gene. This insert was introduced into the replicon construct using an Asco restriction site located in cores directly upstream of the neomycin marker gene. The adaptive mutation at position 1179 (serine to isoleucine) was also introduced (Blight K J, Kolykhalov, A A, Rice, C M, Science 290(5498):1972-1974). A stable cell line constitutively expressing this HCV replicon construct was generated as described above. The luciferase reporter assay was set up as described for the HCV replicon FRET assay with the following modifications. Following 4 days in a 37° C./5% CO₂ incubator, cells were analyzed for Renilla Luciferase activity using the Promega Dual-Glo Luciferase Assay System. Media (100 μl) was removed from each well containing cells. To the remaining 50 μl of media, 50 μl of Dual-Glo Luciferase Reagent was added, and plates rocked for 10 min to 2 h at room temperature. Dual-Glo Stop & Glo Reagent (50 μl) was then added to each well, and plates were rocked again for an additional 10 min to 2 h at room temperature. Plates were read on a Packard TopCount NXT using a luminescence program.

The percentage inhibition was calculated using the formula below:

$$\% control = \frac{\begin{matrix}average luciferase signal in experimental wells \\ ( + compound )\end{matrix}}{\begin{matrix}average luciferase signal in DMSO control wells \\ ( - compound )\end{matrix}}$$

The values were graphed and analyzed using XLfit to obtain the EC₅₀ value.

Representative compounds of the disclosure were assessed in the HCV enzyme assays, HCV replicon cell assay and/or in several of the outlined specificity assays. For example, Compound 2A was found to have an IC₅₀ of 8.9 nanomolar (nM) against the NS3/4A BMS strain in the enzyme assay. Similar potency values were obtained with the published H77 (IC₅₀of 1.4 nM) and J4L6S (IC₅₀ of 1.2 nM) strains. The EC₅₀ value in the replicon FRET assay was 69 nM.

In the specificity assays, the same compound was found to have the following activity: HLE 4.6 μM; PPE>100 μM; Chymotrypsin=2.1 μM; Cathepsin B>100 μM. These results indicate this family of compounds is highly specific for the NS3 protease and many of these members inhibit HCV replicon replication.

The compounds of the current disclosure were tested and found to have activities as follows:

IC₅₀ Activity Range (NS3/4A BMS Strain); A is >0.2 μM; B is 0.02-0.2 μM; C is 4-20 nM.

EC₅₀ Activity Ranges (for compounds tested): A is >1 μM; B is 0.1-1 μM; C is 14-100 nM.

TABLE 2

| Compound Number | IC50 (range or value) | EC50 (range or value) |
|---|---|---|
| 1A | 14.00 nM | 65.26 nM |
| 1B | B | B |
| 2A | C | C |
| 2B | 135 nM | 3.48 μM |
| 3 | 4.55 nM | 14.63 nM |
| 4 | C | C |
| 5 | C | C |

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A compound of formula (I) 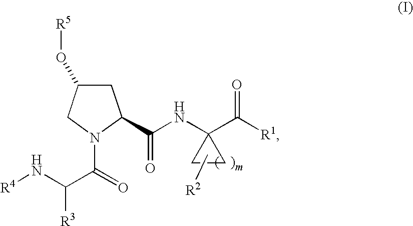
or a pharmaceutically acceptable salt thereof, wherein
m is 1, 2, or 3;
R¹ is selected from hydroxy and —NHSO₂R⁶; wherein R⁶ is selected from alkyl, aryl, cycloalkyl, (cycloalkyl)alkyl, heterocyclyl, and —NR^{a}R^{b}, wherein the alkyl, the cycloalkyl and the cycloalkyl part of the (cycloalkyl)alkyl are optionally substituted with one, two, or three substituents selected from alkenyl, alkoxy, alkoxyalkyl, alkyl, arylalkyl, arylcarbonyl, cyano, cycloalkenlyl, (cycloalkyl)alkyl, halo, haloalkoxy, haloalkyl, and (NR^{e}R^{f})carbonyl;
R² is selected from hydrogen, alkenyl, alkyl, and cycloalkyl, wherein the alkenyl, alkyl, and cycloalkyl are optionally substituted with halo;
R³ is selected from alkenyl, alkoxyalkyl, alkoxycarbonylalkyl, alkyl, arylalkyl, carboxyalkyl, cyanoalkyl, cycloalkyl, (cycloalkyl)alkyl, haloalkoxy, haloalkyl, (heterocyclyl)alkyl, hydroxyalkyl, (NR^{c}R^{d})alkyl, and (NR^{e}R^{f})carbonylalkyl;
R⁴ is selected from phenyl and a five- or six-membered partially or fully unsaturated ring optionally containing one, two, three, or four heteroatoms selected from nitrogen, oxygen, and sulfur; wherein each of the rings is optionally substituted with one, two, three, or four substitutents independently selected from alkoxy, alkoxycarbonyl, alkyl, alkylcarbonyl, alkylsulfanyl, carboxy, cyano, cycloalkyl, cycloalkyloxy, halo, haloalkyl, haloalkoxy, −NR^{c}R^{d}, (NR^{e}R^{f})carbonyl, (NR^{e}R^{f})sulfonyl, and oxo; provided that when R⁴ is a six-membered substituted ring all substituents on the ring other than fluoro must be in the meta and/or para positions relative to the ring's point of attachment to the parent molecular moiety;
R⁵ is selected from alkylcarbonyl, aryl, arylalkyl, arylalkylcarbonyl, arylcarbonyl, heterocyclyl, heterocyclylalkyl, heterocyclylalkylcarbonyl, heterocyclylcarbonyl, and (NR^{g}R^{h})carbonyl, wherein the aryl; the aryl part of the arylalkyl, the arylalkylcarbonyl, and the arylcarbonyl; the heterocycyl; and the heterocyclyl part of the heterocyclylalkyl and the heterocyclylalkylcarbonyl are each optionally substituted with from one to six R⁷ groups; provided that when R⁵ is heterocyclyl the heterocyclyl is other than 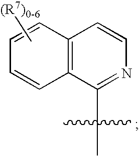
each R⁷ is independently selected from alkoxy, alkoxycarbonyl, alkyl, alkylcarbonyl, aryl, carboxy, cyano, cyanoalkyl, cycloalkyl, halo, haloalkyl, haloalkoxy, heterocyclyl, hydroxy, hydroxyalkyl, nitro, —NR^{c}R^{d}, (NR^{c}R^{d})alkyl, (NR^{c}R^{d})alkoxy, (NR^{e}R^{f})carbonyl, and (NR^{e}R^{f})sulfonyl; or
two adjacent R⁷ groups, together with the carbon atoms to which they are attached, form a four- to seven-membered partially- or fully-unsaturated ring optionally containing one or two heteroatoms independently selected from nitrogen, oxygen, and sulfur, wherein the ring is optionally substituted with one, two, or three groups independently selected from alkoxy, alkyl, cyano, halo, haloalkoxy, and haloalkyl;
R^{a} and R^{b} are independently selected from hydrogen, alkoxy, alkyl, aryl, arylalkyl, cycloalkyl, (cycloalkyl)alkyl, heterocyclyl, and heterocyclylallcyl; or R^{a} and R^{b} together with the nitrogen atom to which they are attached form a four to seven-membered monocyclic heterocyclic ring;
R^{c} and R^{d} are independently selected from hydrogen, alkoxyalkyl, alkoxycarbonyl, alkyl, alkylcarbonyl, arylalkyl, and haloalkyl;
R^{e} and R^{f} are independently selected from hydrogen, alkyl, aryl, arylalkyl, and heterocyclyl; wherein the aryl, the aryl part of the arylalkyl, and the heterocyclyl are optionally substituted with one or two substituents independently selected from alkoxy, alkyl, and halo; and
R^{g} and R^{h} are independently selected from hydrogen, alkyl, aryl, arylalkyl, cycloalkyl, (cycloalkyl)alkyl, heterocyclyl, and heterocyclyl; or R^{g} and R^{h} together with the nitrogen atom to which they are attached form a monocyclic heterocyclic ring wherein the monocyclic heterocyclic ring is optionally fused to a phenyl ring to form a bicyclic system; wherein the monocyclic heterocyclic ring and the bicyclic system are optionally substituted with one, two, or three substituents independently selected from alkoxy, alkyl, halo, haloalkoxy, and haloalkyl.

2. A compound of claim 1, or a pharmaceutically acceptable salt thereof, wherein R¹ is —NHSO₂R⁶.

3. A compound of claim 1, or a pharmaceutically acceptable salt thereof, wherein
m is 1 or 2;
R¹ is —NHSO₂R⁶; wherein R⁶ is selected from alkyl, aryl, cycloalkyl, (cycloalkyl)alkyl, heterocyclyl, and —NR^{a}R^{b}, wherein the alkyl, the cycloalkyl and the cycloalkyl part of the (cycloalkyl)alkyl are optionally substituted with one, two, or three substituents selected from alkenyl, alkoxy, alkoxyalkyl, alkyl, arylalkyl, arylcarbonyl, cyano, cycloalkenyl, (cycloalkyl)alkyl, halo, haloalkoxy, haloalkyl, and (NR^{e}R^{f})carbonyl;
R² is selected from alkenyl, alkyl, and cycloalkyl, wherein the alkenyl, alkyl, and cycloalkyl are optionally substituted with halo;
R³ is selected from alkenyl and alkyl;
R⁴ is selected from phenyl and a five- or six-membered partially or fully unsaturated ring optionally containing one, two, three, or four heteroatoms selected from nitrogen, oxygen, and sulfur; wherein each of the rings is optionally substituted with one, two, three, or four substitutents independently selected from alkoxy, alkoxycarbonyl, alkyl, alkylcarbonyl, alkylsulfanyl, carboxy, cyano, cycloalkyl, cycloalkyloxy, halo, haloalkyl, haloalicoxy, —NR^{c}R^{d}, (NR^{e}R^{f})carbonyl, (NR^{e}R^{f})sulfonyl, and oxo; provided that when R⁴ is a six-membered substituted ring all substituents on the ring other than fluoro must be in the meta and/or para positions relative to the ring's point of attachment to the parent molecular moiety;
R⁵ is selected from alkylcarbonyl, aryl, arylalkyl, arylalkylcarbonyl, arylcarbonyl, heterocyclyl, heterocyclylalkyl, heterocyclylalkylcarbonyl, heterocyclylcarbonyl, and (NR^{g}R^{h})carbonyl, wherein the aryl; the aryl part of the arylalkyl, the arylalkylcarbonyl, and the arylcarbonyl; the heterocycyl; and the heterocyclyl part of the heterocyclylalkyl and the heterocyclylalkylcarbonyl are each optionally substituted with from one to six R⁷ groups; provided that when R⁵ is heterocyclyl the heterocyclyl is other than 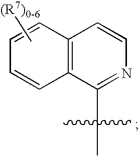
each R⁷ is independently selected from alkoxy, alkoxycarbonyl, alkyl, alkylcarbonyl, aryl, carboxy, cyano, cyanoalkyl, cycloalkyl, halo, haloalkyl, haloalkoxy, heterocyclyl, hydroxy, hydroxyalkyl, nitro, —NR^{c}R^{d}, (NR^{c}R^{d})alkyl, (NR^{c}R^{d})alkoxy, (NR^{e}R^{f})carbonyl, and (NR^{e}R^{f})sulfonyl; or
two adjacent R⁷ groups, together with the carbon atoms to which they are attached, form a four- to seven-membered partially- or fully-unsaturated ring optionally containing one or two heteroatoms independently selected from nitrogen, oxygen, and sulfur, wherein the ring is optionally substituted with one, two, or three groups independently selected from alkoxy, alkyl, cyano, halo, haloalkoxy, and haloalkyl;
R^{a} and R^{b} are independently selected from hydrogen, alkoxy, alkyl, aryl, arylalkyl, cycloalkyl, (cycloalkyl)alkyl, heterocyclyl, and heterocyclylalkyl; or R^{a} and R^{b} together with the nitrogen atom to which they are attached form a four- to seven-membered monocyclic heterocyclic ring;
R^{c} and R^{d} are independently selected from hydrogen, alkoxyalkyl, alkoxycarbonyl, alkyl, alkylcarbonyl, arylalkyl, and haloalkyl;
R^{e} and R^{f} are independently selected from hydrogen, alkyl, aryl, arylalkyl, and heterocyclyl; wherein the aryl, the aryl part of the arylalkyl, and the heterocyclyl are optionally substituted with one or two substituents independently selected from alkoxy, alkyl, and halo; and
R^{g} and R^{h} are independently selected from hydrogen, alkyl, aryl, arylalkyl, cycloalkyl, (cycloalkyl)alkyl, heterocyclyl, and heterocyclyl; or R^{g} and R^{h} together with the nitrogen atom to which they are attached form a monocyclic heterocyclic ring wherein the monocyclic heterocyclic ring is optionally fused to a phenyl ring to form a bicyclic system; wherein the monocyclic heterocyclic ring and the bicyclic system are optionally substituted with one, two, or three substituents independently selected from alkoxy, alkyl, halo, haloalkoxy, and haloalkyl.

4. A compound of claim 3, or a pharmaceutically acceptable salt thereof, wherein R⁶ is unsubstituted cycloalkyl.

5. A compound of claim 1, or a pharmaceutically acceptable salt thereof, wherein
m is 1;
R¹ is —NHSO₂R⁶; wherein R⁶ is unsubstituted cycloalkyl;
R² is alkenyl;
R³ is alkyl;
R⁴ is selected from phenyl and a fives or six-membered partially or fully unsaturated ring optionally containing one, two, three, or four heteroatoms selected from nitrogen, oxygen, and sulfur; wherein each of the rings is optionally substituted with one, two, or three substitutents independently selected from alkoxy, alkoxycarbonyl, alkyl, alkylcarbonyl, carboxy, cyano, cycloalkyl, cycloalkyloxy, halo, haloalkyl, haloalkoxy, —NR^{c}R^{d}, (NR^{e}R^{f})carbonyl, (R^{e}R^{f})sulfonyl, and oxo; provided that when R⁴ is a six-membered substituted ring all substituents on the ring other than fluoro must be in the meta and/or para positions relative to the ring's point of attachment to the parent molecular moiety;
R⁵ is selected from heterocyclyl and NR^{g}R^{h})carbonyl, wherein the heterocycyl is optionally substituted with from one to six R⁷ groups; provided that R⁵ is other than 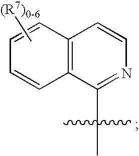
each R⁷ is independently selected from alkoxy, aryl, and heterocyclyl;
R^{c} and R^{d} are independently selected from hydrogen, alkoxycarbonyl, alkyl, alkylcarbonyl, and arylalkyl;
R^{e} and R^{f} are independently selected from hydrogen, alkyl, aryl, and arylalkyl; and
R^{g} and R^{h} together with the nitrogen atom to which they are attached form a monocyclic heterocyclic ring fused to a phenyl ring to form a bicyclic system; wherein the bicyclic system is substituted with a halo group.

6. A compound of claim 5, or a pharmaceutically acceptable salt thereof, wherein R⁴ is six-membered unsaturated ring containing one nitrogen atom wherein the ring is optionally substituted with one, two, or three substitutents independently selected from alkoxy, alkoxycarbonyl, alkyl, alkylcarbonyl, carboxy, cyano, cycloalkyl, cycloalkyloxy, halo, haloalkyl, haloalkoxy, —NR^{c}R^{d}, (NR^{e}R^{f})carbonyl, (NR^{e}R^{f})sulfonyl, and oxo; provided that all substituents on the ring other than fluoro must be in the meta and/or para positions relative to the ring's point of attachment to the parent molecular moiety.

7. A compound of claim 5, or a pharmaceutically acceptable salt thereof, wherein R⁴ is five-membered unsaturated ring containing one nitrogen atom and one sulfur atom, wherein the ring is optionally substituted with one, two, or three substitutents independently selected from alkoxy, alkoxycarbonyl, alkyl, alkylcarbonyl, carboxy, cyano, cycloalkyl, cycloalkyloxy, halo, haloalkyl, haloalkoxy, —NR^{c}R^{d}, (NR^{e}R^{f})carbonyl, (NR^{e}R^{f})sulfonyl, and oxo.

8. A compound selected from

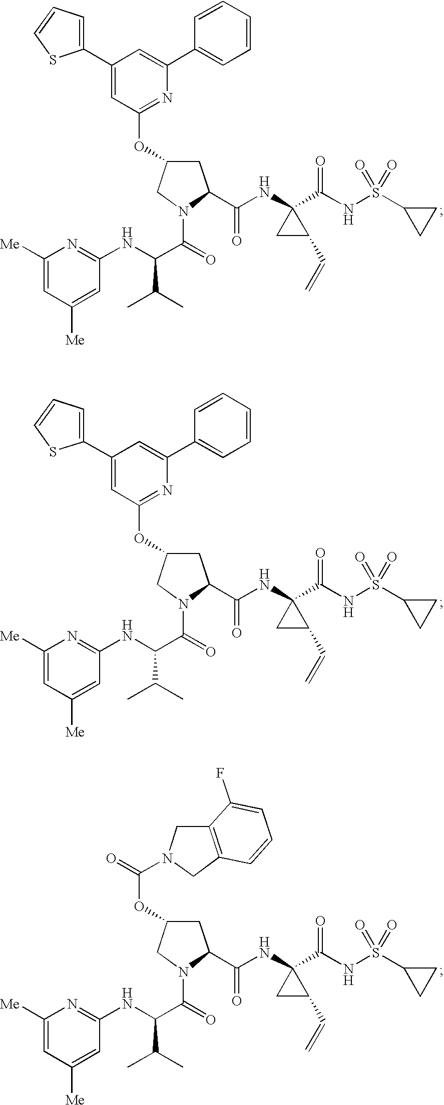

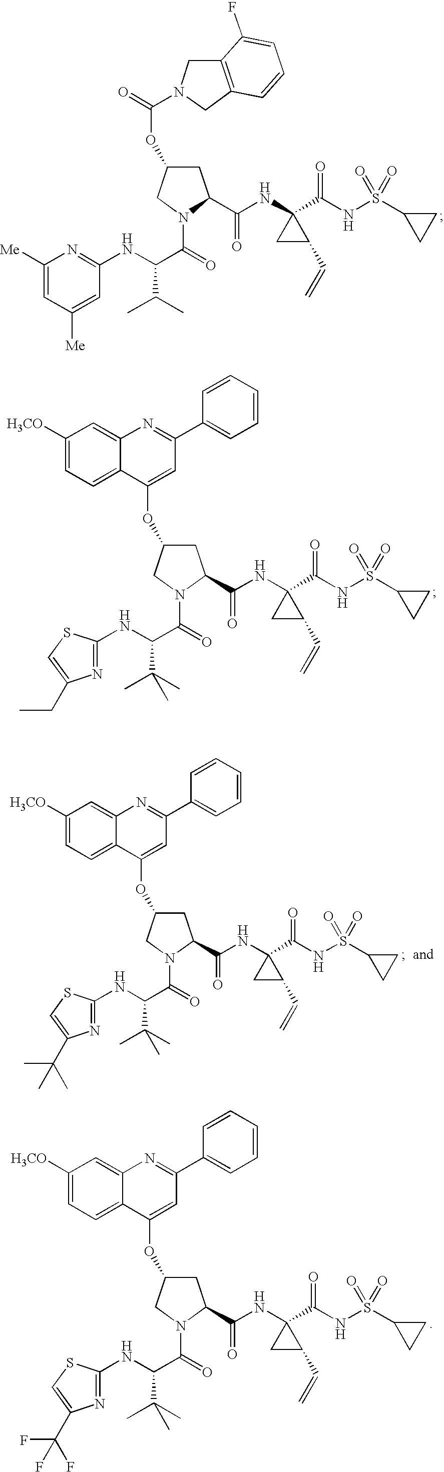

9. A composition comprising the compound of claim 1, or a pharmaceutically acceptable salt thereof, and a pharmaceutically acceptable carrier.

10. The composition of claim 9 further comprising at least one additional compound having anti-HCV activity.

11. The composition of claim 10 wherein at least one of the additional compounds is an interferon or a ribavirin.

12. The composition of claim 11 wherein the interferon is selected from interferon alpha 2B, pegylated interferon alpha, consensus interferon, interferon alpha 2A, and lymphoblastiod interferon tau.

13. The composition of claim 10 wherein at least one of the additional compounds is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, ainantadine, and rimantadine.

14. The composition of claim 10 wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, fHCV NS5A protein, and IMPDH for the treatment of an HCV infection.

15. A method of treating an HCV infection in a patient, comprising admninistering to the patient a therapeutically effective amount of a compound of claim 1, or a pharmaceutically acceptable salt thereof.

16. The method of claim 15 further comprising administering at least one additional compounds having anti-HCV activity prior to, after, or simultaneously with the compound of claim 1, or a pharmaceutically acceptable salt thereof.

17. The method of claim 16 wherein at least one of the additional compounds is an interferon or a ribavirin.

18. The method of claim 17 wherein the interferon is selected from interferon alpha 2B, pegylated interferon alpha, consensus interferon, interferon alpha 2A, and lymphoblastiod interferon tau.

19. The method of claim 16 wherein at least one of the additional compounds is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

20. The method of claim 16 wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, HCV NS5A protein, and IMPDH for the treatment of an HCV infection.
